(19) United States
(12) Patent Application Publication
(10) Pub. No.: US 2018/0188270 A1
(43) Pub. Date: Jul. 5, 2018

(54) DIAGNOSTIC TEST FOR ALZHEIMER'S DISEASE BASED ON IDENTIFICATION OF A PROTEOLYTIC PATHWAY

(71) Applicant: Health Research, Inc., Buffalo, NY (US)

(72) Inventors: Yuesheng ZHANG, Orchard Park, NY (US); Lu YANG, Buffalo, NY (US); Yun LI, Orchard Park, NY (US); Arup BHATTACHARYA, Buffalo, NY (US)

(21) Appl. No.: 15/740,616

(22) PCT Filed: Jun. 30, 2016
(86) PCT No.: PCT/US16/40423

§ 371 Date: Dec. 28, 2017

Related U.S. Application Data
(60) Provisional application No. 62/186,439, filed on Jun. 30, 2015.

(51) Int. Cl. G01N 33/68

(52) U.S. Cl. CPC G01N 33/6896; G01N 2333/4709; G01N 2800/52; G01N 2800/2821; G01N 2800/7047; G01N 2400/40

(57) ABSTRACT

A method for detecting circulating levels of beta amyloid peptides in an individual by administration of one or more inhibitors of an extracellular proteolytic pathway in blood and detection of the beta amyloid peptides in the blood or a fraction thereof. An example of an inhibitor is an anticoagulant. Levels of desired beta amyloid peptides such as Aβ1-40 and Aβ1-42 can be determined in the blood samples collected from the individuals.

### CROSS-REFERENCE TO RELATED APPLICATIONS

This application claims priority to U.S. provisional application No. 62/186,439, filed on Jun. 30, 2015, the disclosure of which is incorporated herein by reference.

### STATEMENT REGARDING FEDERALLY SPONSORED RESEARCH

This invention was made with government support under grant no. R01CA164574 awarded by the National Institutes of Health. The government has certain rights in the invention.

### BACKGROUND OF THE DISCLOSURE

Amyloid beta (Aβ) is the major component of senile plaque, one of the hallmarks of Alzheimer disease (AD) pathology and results from proteolytic cleavage of amyloid precursor protein (APP). Different Aβ isoforms may be generated, depending on the cleavage site, but Aβ1-40 and Aβ1-42 are the main isoforms. APP gene mutation or increased gene dosage (Down syndrome, DS) causes increased Aβ production in the brain, leading to brain Aβ accumulation and aggregation. Brain Aβ is cleared to the cerebrospinal fluid and plasma. Both decreased Aβ clearance and increased Aβ production in the brain have been reported in sporadic AD. Aβ1-42 is generated from proteolytic cleavage of cell-membrane-bound APP by secretases and is present in the plasma at low levels. Aβ1-40 is also generated by proteolytic cleavage of APP. Aβ1-40 and Aβ1-42 are considered key drivers of AD. Plasma levels of Aβ1-42 and Aβ1-40 may increase up to 2-3 fold in familial AD and DS, but show little increase, if any, in sporadic AD, and are not currently considered an AD biomarker. Accordingly, there is an unmet need in the area of blood-based biomarkers of AD.

### SUMMARY OF THE DISCLOSURE

In this disclosure, we describe a proteolysis pathway in the blood that detects and degrades proteins and polypeptides. We demonstrate the presence and mechanism of this pathway using the examples of prolidase (PEPD) and SRC, and demonstrate that this pathway is also responsible for rapid degradation of APP products such as Aβ1-42 and Aβ1-40.

We demonstrate that upon entering the blood circulation, certain proteins can rapidly activate intrinsic coagulation cascade by binding and activating factor XII (FXII), which then activates the intrinsic coagulation cascade, including factors prekallikrein (PK), high molecular weight kininogen (HMWK), factor XI (FXI) and factor IX (FIX), and part of the common coagulation cascade, including factors X (FX) and II (FII), which in turn activates factor VII (FVII), a key factor of the extrinsic coagulation cascade (FIG. 12). Activated FVII (FVIIa) rapidly degrades the proteins. Examples of proteins that undergo degradation via this pathway include prolidase, SRC, and Aβ peptides (such as Aβ1-42 and Aβ1-40). Blood coagulation activity likely also increases due to the activation of this pathway.

These results reveal the latent functions of the plasma coagulation factors and identify FXII and FVIIa as the sensor/initiator and executioner respectively of a multi-component, multi-step proteolysis pathway in the plasma. Our Aβ1-42 and Aβ1-40 results are particularly noteworthy, as they are key drivers of Alzheimer disease (AD). We further show that temporary pharmacologic inhibition of the proteolysis pathway blocks Aβ1-42 and Aβ1-40 degradation in the plasma.

Based on our findings, in one aspect, this disclosure provides a method for detection of Aβ peptides in the blood or fractions thereof. The Aβ peptides detected in the blood include, but are not limited to, Aβ1-40 and Aβ1-42.

Such detection can aid in the diagnosis of conditions in which there is increase in Aβ containing plaques in the brain or elsewhere. The method can also be used for monitoring the status or progression of conditions involving Aβ containing plaque formation, and for monitoring the status or progression of therapies relating to treating such conditions. The method comprises inhibition of the proteolytic degradation pathway in an individual (such as by administration to the individual of an inhibitor of the proteolytic degradation pathway) thereby allowing accumulation and therefore detection of Aβ peptides in blood (such as Aβ1-42 and Aβ1-40). In one embodiment, the inhibition of proteolytic degradation pathway can be carried out after collection of blood from an individual. The method can be used for detection of AD, for following progression of AD, and/or for monitoring therapeutic efficacy. The AD may be familial or sporadic.

In one aspect, this disclosure provides kits for the detection of AD. The kits comprise reagents for inhibition of the proteolytic pathway, and reagents for detection of Aβ peptides (such as Aβ1-42 and/or Aβ1-40) in blood or a fraction thereof (such as serum or plasma).

### BRIEF DESCRIPTION OF THE FIGURES

FIG. 1. PEPD degradation in the plasma. (A) Plasma PEPD concentrations in wild type (WT) mice treated with enoxaparin (EP) and/or recombinant human PEPD (hPEPD). EP (2.5 mg/kg) was given to mice i.p. once daily for 5 days. hPEPD (0.2 or 10 mg/kg) or vehicle was given to mice i.p. alone or 1 h after the last EP dose. Blood samples were collected from the mice at 1 or 24 h after hPEPD/vehicle treatment for measurement of plasma PEPD by enzyme-linked immunosorbent assay (ELISA). (B-J) Changes in plasma coagulation factors in WT mice treated with EP and/or hPEPD. Mice were treated with EP as described in A. hPEPD (0.2 mg/kg) or vehicle was given to mice i.p. alone or 1 h after the last EP dose; blood samples were collected from the mice at 6 h after hPEPD/vehicle treatment; 7.5 μl plasma per sample was analyzed by immunoblotting (IB). Arrows indicate cleaved fragments. Plasma level of coagulation factor I (FI, also known as fibrinogen)) was measured by ELISA. Error bars in A and J indicate SD (n=3). Data were analyzed by two-way ANOVA in A or one-way ANOVA in J, followed by Tukey multiple comparisons test. Data in A were log transformed before ANOVA. * P<0.05; **** P<0.0001.

FIG. 2. hPEPD degradation by FVIIa. (A) hPEPD (90 nM) was incubated with activated human coagulation factor VII (FVIIa) (10 nM) plus human tissue factor (TF) (10 nM) in CaCl₂-containing phosphate-buffered saline (PBS) at room temperature (RT), and then measured at different times for remaining hPEPD by enzymatic activity analysis. (B) hPEPD at 10 nM (+̂̂), 40 nM (+̂) or 90 nM (+) was incubated with FVIIa (10 nM) plus TF (10 nM) in CaCl₂-containing PBS at RT for 60 min; remaining hPEPD was measured by enzymatic activity analysis. (C) hPEPD (90 nM) was incubated alone or with FVIIa (10 nM) plus TF (10 nM) in CaCl₂-containing PBS at RT, and then separated by sodium dodecyl sulfate polyacrylamide gel electrophoresis (SDS-PAGE) and stained by silver. FVIIa and TF were also incubated without hPEPD, as a control. (D) hPEPD (90 nM) was incubated with FVIIa (10 nM) with or without TF (10 nM) in CaCl₂-containing PBS at RT, followed by measurement of remaining hPEPD enzymatic activity. (E) hPEPD (90 nM) was incubated alone, with human coagulation factor VII (FVII) (10 nM), with FVII (10 nM) plus TF (10 nM), with FVII (10 nM) plus activated human coagulation factor II (FIIa) (100 nM), or with FVII (10 nM) plus TF (10 nM) and FIIa (100 nM) in CaCl₂-containing PBS at RT for 24 h, followed by measurement of remaining hPEPD by enzymatic analysis. Error bars in A, B, D and E indicate SD (n=3). Data in B, D and E were analyzed by one-way ANOVA, followed by Tukey multiple comparisons test. * P<0.05; ** P<0.01; ****P<0.0001.

FIG. 3. hPEPD binds to PRD in FXII and activates FXII. (A, B) hPEPD or a mutant (40 nM) was incubated with a blood coagulation factor (0.5 μM) in PBS at 37° C. for 2 h, followed by immunoprecipitation (IP) and IB. (C) human coagulation factor XII (FXII) (0.97 nM) was incubated with hPEPD or a mutant in ZnCl₂-containing PBS at RT; FXII activation was measured by a chromogenic assay. Error bars indicate SD (n=3). (D) hPEPD (40 nM) was incubated with FXII (0.5 μM) in PBS (0.1 ml volume) with or without ZnCl₂ (15 μM) at RT, followed by IB (7.5 μl per sample). (E) FXII or a mutant (0.5 μM) was incubated with hPEPD (40 nM) in PBS at 37° C. for 2 h, followed by IP and IB.

FIG. 4. FXII initiates plasma PEPD degradation. (A) Plasma PEPD concentrations in control mice and mice treated with EP, hPEPD, or EP plus hPEPD. EP (2.5 mg/kg) was given to mice i.p. once daily for 5 days; hPEPD (0.2 mg/kg) or vehicle was given to mice i.p. alone or at 1 h after the last EP dose. Blood samples were collected from the mice at 6 h after hPEPD/vehicle treatment for measurement of plasma PEPD level by ELISA. Error bars indicate SD (n=3). Data were log transformed before two-way ANOVA, followed by Tukey multiple comparisons test. *P<0.05; **** P<0.0001. (B) Changes in plasma coagulation factors in mice treated as described in A; 7.5 μl plasma per sample was analyzed by IB. Arrows indicate cleaved fragments.

FIG. 5. Plasma PEPD is degraded exclusively by FVIIa. (A, B) hPEPD (9.2 pmol) was incubated at RT with plasma (100 μl) with or without FIIa or activated human coagulation factor X (FXa) (10 pmol) for 24 h and after centrifugation to remove the precipitates, remaining PEPD was measured by enzymatic activity analysis. The precipitates were washed by PBS and checked for presence of hPEPD by IB, using activated human coagulation factor XIII (FXIIIa) and pure hPEPD as a binding control and standard, respectively. (C) Plasma samples from untreated WT mice were incubated with an antibody that binds to both FVII and FVIIa; the immunocomplexes were pulled down with protein A sepharose. The supernatant fraction along with a regular plasma sample was analyzed for FVII/FVIIa level by IB. FVIIa was undetectable in the samples. (D) hPEPD (9.2 pmol) was incubated with regular plasma or FVII/FVIIa-depleted plasma (100 μl) in the absence or presence of FIIa (10 pmol) at RT for 24 h; remaining hPEPD was measured by hPEPD enzymatic activity. Notably, plasma samples used in B and C were prepared from blood drawn from mice without an anticoagulant. (E, F) Plasma PEPD and coagulation factors in control mice and mice at 6 h after hPEPD treatment; PEPD concentration was measured by ELISA, and coagulation factors were analyzed by IB (7.5 μl plasma per sample). Arrows indicate cleaved fragments. Error bars in A, D and E indicate SD (n=3). Data were analyzed by one-way ANOVA in A or two-way ANOVA in D and E, followed by Tukey multiple comparisons test. Data in E were log transformed before ANOVA. * P<0.05; **** P<0.0001.

FIG. 6. SRC binds to PRD in FXII and activates FXII but is degraded by FVIIa. (A) mouse SRC (mSRC) (40 nM) was incubated with FXII or a mutant (0.5 μM) in PBS at 37° C. for 2 h, followed by IP and IB. (B) mSRC (0, 20 and 200 nM) was incubated with FXII (0.97 nM) in ZnCl₂-containing PBS at RT; FXII activation was measured by a chromogenic assay. Each value is mean±SD (n=3). (C) mSRC (0.17 μM) was incubated alone or with FVIIa (10 nM) and TF (10 nM) in CaCl₂-containing PBS at RT for indicated times, separated by SDS-PAGE and stained by silver. FVIIa and TF were incubated without mSRC as a control.

FIG. 7. The FXII-FVII proteolysis pathway detects and degrades SRC. WT mice, FXII^{-/-} mice and FVII^{tTA/tTA} mice were treated with vehicle or mSRC i.p.; blood samples were collected from the mice at 6 h after treatment. (A) Plasma concentrations of mSRC, measured by ELISA. (B-J) Changes in plasma coagulation factors, measured by IB (7.5 μl plasma per sample) or ELISA. Arrows indicate cleaved fragments. Error bars in A and J indicate SD (n=3). Data were analyzed by two-way ANOVA in A or one-way ANOVA in J, followed by Tukey multiple comparisons test. Data in A were log transformed before ANOVA, **** P<0.0001; ns, not significant.

FIG. 8. Aβ1-42 binds to FN2D in FXII and activates FXII but is degraded by FVIIa. (A) Human Aβ1-42 (hAβ1-42) (0, 0.05, 0.5 and 11.1 μM) was incubated with FXII (0.97 nM) in ZnCl₂-containing PBS at RT; FXII activation was measured by a chromogenic assay. Each value is mean±SD (n=3). (B) hAβ1-42 (200 nM) was incubated with FXII or a mutant (20 nM) in PBS at RT; hAβ1-42 aggregated during the incubation. FXII or its mutant remaining in solution (S) or binding to hAβ1-42 aggregates (A), the latter of which was re-dissolved in 2% SDS, were measured by IB. (C) hAβ1-42 (2.2 μM) was incubated alone, or with FVIIa (10 nM) and TF (10 nM) in CaCl₂-containing PBS at RT for indicated times; the aggregates were re-dissolved in 2% SDS and mixed with the supernatant fraction, separated by SDS-PAGE and stained by silver. FVIIa and TF were incubated without hAβ1-42, as a control.

FIG. 9. The FXII-FVII proteolysis pathway detects and degrades Aβ1-42. WT mice, FXII^{-/-} mice and FVII^{tTA/tTA} mice were treated with vehicle or hAβ1-42 i.p.; blood samples were collected from the mice at 6 h after treatment. (A) Plasma concentrations of Aβ1-42, measured by ELISA. (B-J) Changes in plasma coagulation factors, measured by IB (7.5 μl plasma per sample) or ELISA. Arrows indicate cleaved fragments. Error bars in A and J indicate SD (n=3). Data were analyzed by two-way ANOVA in A or one-way ANOVA in J, followed by Tukey multiple comparisons test. Data in A were log transformed before ANOVA, **** P<0.0001; ns, not significant.

FIG. 10. EP inhibits plasma Aβ1-42 degradation and prevents Aβ1-42 from activating FX, FII and FVII. (A) Plasma Aβ1-42 concentrations in control mice and mice treated with EP, hAβ1-42, or EP plus hAβ1-42. EP (2.5 mg/kg) was given to WT mice i.p. once daily for 5 days. hAβ1-42 (40 μg/kg) or vehicle was given to mice i.p. alone or 1 h after the last EP dose. Blood samples were collected from the mice at 6 h after hAβ1-42/vehicle treatment for measurement of plasma Aβ1-42 by ELISA. Error bars indicate SD (n=3). Data were log transformed before one-way ANOVA, followed by Tukey multiple comparisons test. **** P<0.0001. (B-I) Changes in plasma coagulation factors in control mice and mice treated with EP, hAβ1-42, or EP plus hAβ1-42 as described in A; 7.5 μl plasma per sample was analyzed by IB. Arrows indicate cleaved fragments.

FIG. 11. The FXII-FVII proteolysis pathway responds to tissue injury. Mice were treated i.p. with vehicle (filled circle: corn oil for CCl₄; filled square: PBS for hPEPD), CCl₄ (0.5 g/kg) or hPEPD (4 mg/kg); blood samples and various organs were collected 24 h later. (A-C) Plasma levels of mouse PEPD (mPEPD) and/or hPEPD, mSRC and mouse Aβ1-42 (mAβ1-42) were measured by ELISA. Error bars indicate SD (n=3); data were log transformed before two-way ANOVA, followed by Tukey multiple comparisons test. ** P<0.01; *** P<0.001; **** P<0.0001. (D) Tissue levels of ERBB1 and ERBB2 and their phosphorylation status were measured by IB. GAPDH is a loading control. Each lane represents a sample from a different mouse.

FIG. 12. The FXII-FVII proteolysis pathway that detects and degrades PEPD, SRC and Aβ1-42, and its inhibition by EP. PEPD, SRC or Aβ1-42 activates FXII by binding to a different domain in FXII. FXII activation leads to activation of FX and FII, which in turn activates FVII, and activated FVII degrades PEPD, SRC and Aβ1-42. EP blocks the degradation of PEPD, SRC and Aβ1-42 in the plasma by binding and activating antithrombin III (AT), which inhibits several coagulation factors in the proteolysis pathway. The “↓” and “T” symbols indicate activation and inhibition, respectively.

FIG. 13. Characterization of mPEPD, and its activation of the proteolysis pathway. (A) mPEPD, measured by IB and compared to hPEPD. (B) mPEPD, measured by SDS-PAGE followed by silver staining and compared to hPEPD. (C, D) Plasma levels of mPEPD and fibrinogen (FI) in mice treated with mPEPD, measured by ELISA. Error bars indicate SD (n=3). Data were analyzed by two-way ANOVA, followed by Tukey multiple comparisons test. Data in C were log transformed before ANOVA. **** P<0.0001. (E-L) Effect of mPEPD on plasma coagulation factors in mice, measured by IB. Plasma samples were obtained from WT mice and FXII^{-/-} mice at 6 h after i.p. injection of solvent or mPEPD (0.2 mg/kg). Each lane represents 7.5 ml of plasma sample. Arrows indicate cleaved fragments.

FIG. 14. No effect of FXa and FIIa on hPEPD stability. hPEPD (90 nM) was incubated alone, with FXa (100 nM) or FIIa (100 nM) in PBS containing 5 mM CaCl₂ (total volume of 0.1 ml) for a specific time at RT. The incubated samples were analyzed for remaining hPEPD by IB (A, C) or by measurement of hPEPD enzymatic activity (B, D). Error bars indicate SD (n=3).

FIG. 15. Sequence information of hPEPD and its mutants. Each protein has 6xHis tagged to its carboxy terminus.

FIG. 16. Characterization of FXII and its mutants. (A) Location of various domains in human FXII. (B) Sequence information on FXII mutants. (C) Comparison of relative molecular size of recombinant FXII and its mutants. FXII and its mutants were generated in CHO-K1 cells, purified by NI-NTA agarose chromatography, and compared for molecular size by IB, using an antibody binding to either their C-termini or C-terminal His tag. (D) Purified FXII and its mutants were resolved by SDS-PAGE and stained by silver to assess purity.

FIG. 17. Plasma levels of FXII and FVII in WT mice, FXII^{-/-} mice and FVII^{tTA/tTA} mice. Plasma samples (7.5 ml each) were analyzed by IB.

FIG. 18. hPEPD does not directly activate FX, FII or FVII. hPEPD (40 nM) was incubated with FX, FII or FVII (0.5 mM for each factor) in the presence of 5 mM CaCl₂ in PBS at RT for 24 h and then analyzed by IB. FXa, FIIa and FVIIa were used as positive controls in the experiments.

FIG. 19. Changes in plasma levels of AST and ALT and in liver weight after treatment with CCl₄. WT mice and FXII^{-/-} mice were treated i.p. with vehicle or CCl₄ at 0.5 g/kg; 24 h later, the mice were killed, plasma samples were prepared and measured for aspartate aminotransferase (AST) and alanine aminotransferase (ALT) activities, and the livers were weighed. Error bars indicate SD (n=3). The data were analyzed by two-way ANOVA, followed by Tukey multiple comparisons test. *** P<0.001; **** P<0.0001; ns, not significant.

FIG. 20. Background activation of the FXII-FVII pathway factors. Plasma samples (7.5 ml each) from untreated WT mice, FXII^{-/-} mice and FVII^{tTA/tTA} mice were analyzed by IB. Each lane represents one mouse. Note: All the films were highly overexposed in order to detect the minute levels of cleaved fragment(s) of each factor. Arrows indicate cleaved fragments.

FIG. 21. hAβ1-40 activates FXII and is degraded by FVIIa. (A) hAβ1-40 at indicated concentrations was incubated with human FXII (0.97 nM) in ZnCl₂-containing PBS at room temperature and FXII activation was measured by a chromogenic assay. Each value is mean±SD (n=3). (B) hAβ1-40 (2.2 μM) was incubated alone or with FVIIa (10 nM) plus human TF (10 nM) in CaCl₂-containing PBS at room temperature for indicated times, followed by SDS-PAGE.

FIG. 22. EP elevates plasma level of hAβ1-40. Male C57BL/6 mice (7-8 weeks of age) were treated with vehicle or EP (0.5 mg/kg) i.p. once daily for 5 days. One hour after the fifth dose of vehicle/EP, hAβ1-40 (40 μg/kg) was administered i.p. to the mice, and 6 h later, blood was collected from the mice. Plasma levels of hAβ1-40 were measured by ELISA. Error bars indicate SD (n=3).

FIG. 23. EP elevates plasma level of Aβ1-40/Aβ1-42 in AD mice. WT mice and AD mice at 1 month of age and 3 months of age were treated with vehicle or EP (0.5 mg/kg) i.p. once daily for 5 days. Blood was drawn from the mice 6 h after the final treatment and measured by ELISA for total plasma level of Aβ1-40 and Aβ1-42. Error bars represent SD (n=3). Different calculations of the difference in plasma levels of Aβ-1-40 and Aβ1-42 between WT mice and AD mice are also shown.

FIG. 24. Activation of coagulation proteases in J20 AD mice, but EP inhibits the activation of coagulation factors FX, FII and FVII. AD mice and their normal counterparts at 1 month of age were treated with vehicle or EP (0.5 mg/kg) i.p. once daily for 5 days. Blood were drawn from the mice 6 h after the last vehicle/EP dose, assayed for coagulation proteases by western blotting. Arrows indicate cleaved fragments.

FIG. 25. Activation of coagulation proteases in J20 AD mice, but EP inhibits the activation of coagulation factors FX, FII and FVII. AD mice and their normal counterparts at 3 months of age were treated with vehicle or EP (0.5 mg/kg) i.p. once daily for 5 days. Blood were drawn from the mice 6 h after the last vehicle/EP dose, assayed for coagulation proteases by western blotting. Arrows indicate cleaved fragments.

FIG. 26. Effects of oral anticoagulants on plasma level of hAβ1-42. Male C57BL/6 mice (7-8 weeks of age) were treated by oral intubation with vehicle, warfarin (1 or 3 mg/kg), rivaroxaban (10 or 20 mg/kg) or dabigatran (22.5 or 45 mg/kg) once daily for 5 days. One hour after the fifth dose, hAβ1-42 (40 μg/kg) was administered i.p. to the mice, and 6 h later, blood was collected from the mice. Plasma levels of hAβ1-42 were measured by ELISA. Error bars indicate SD (n=3).

### DESCRIPTION OF THE DISCLOSURE

The present disclosure is based on the identification of an extracellular protein degradation pathway in the blood that results in the degradation of certain proteins in circulation. The present disclosure provides a method for inhibition of the degradation pathway so as to inhibit the degradation of certain circulating proteins or polypeptides thereby allowing the proteins to have prolonged action or to allow the proteins to accumulate to make them amenable to detection.

For example, the disclosure provides a method for detecting a condition in which plaques in the brain (or elsewhere) containing amyloid plaque proteins are increased. The plaques may be senile plaques associated with AD. The method comprises: administering to an individual an inhibitor of proteolytic pathway that occurs in blood; after a suitable period of time, during or following administration of the inhibitor, collecting a biological fluid sample from the individual; and detecting the level of one or more Aβ peptides (such as Aβ1-42, Aβ1-40 or other Aβ peptides) in the biological fluid sample collected from the individual, or in a fractionated portion of the biological fluid sample (such as plasma or serum prepared from the blood). It is considered the inhibitor blocks or inhibits the degradation of Aβ peptides thereby allowing detection of increased levels of these peptides in the sample. An increase in the levels of these peptides compared to a reference control is indication of increased Aβ plaques in the brain (or elsewhere) of the individual. Increased plaques may identify the individual as being at risk of developing AD or having AD.

The terms “beta amyloid peptides” or “Aβ peptides” or “Aβ” are used interchangeably and refer to peptide fragments of APP which are a few amino acids to 43 amino acids in length. For example, the peptide fragments can be 10 to 43 amino acids in length. The peptides are generated in vivo as cleavage products of APP by two proteases, β-secretase and γ-secretase. Examples include Aβ1-40 and Aβ1-42.

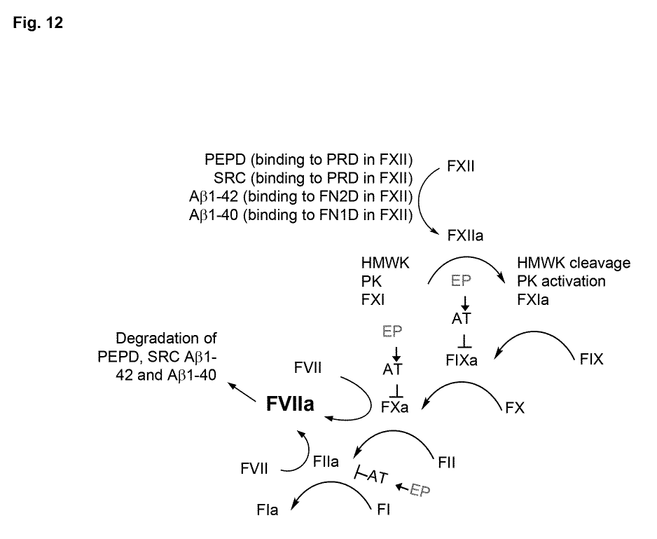

An example of an inhibitor of the blood proteolytic pathway is an anticoagulant. The anticoagulant can be a low molecular weight heparin (LMWH), heparin, warfarin or any other molecule that inhibits one or more steps of the blood proteolytic pathway (illustrated in FIG. 12). For example, peptidemimetics of the molecules involved in the blood proteolytic pathway or antibodies or fragments thereof that inhibit one or more steps of the blood peroteolytic pathway can be used. Low molecular weight heparins can be derived from unfractionated heparin and generally have an average molecular weight of 10,000 or less. For example, low molecular weight heparin can have an average molecular weight of from 3,000 to 8,000.

While not intending to be bound by any particular theory, it is considered that the FXII-FVII proteolysis pathway serves to maintain a low plasma level of proteins or polypeptides such as Aβ1-40, Aβ1-42, PEPD, SRC and others during conditions such as tissue injury In the case of PEPD which is a ligand of ERBB1 and ERBB2, the proteolysis pathway serves to minimize ligand-induced impact on the receptors (ERBB1 and ERBB2) in various tissues (heart, kidney and liver), including inhibition of both receptor tyrosine phosphorylation and receptor down regulation. These results indicate that the FXII-FVII proteolysis pathway serves as a protective mechanism during tissue injury or exogenous insult.

SRC is an intracellular non-receptor tyrosine kinase, but is not known to play any physiological role in the plasma. Its presence in the plasma likely results from leakage from damaged cells and tissues. Degradation of plasma SRC by the FXII-FVII proteolysis pathway shows an example of removal of a nonfunctional protein by the proteolysis pathway.

Our finding that Aβ1-42 and Aβ1-40 activate and then are degraded by the FXII-FVII proteolysis pathway suggests that this pathway may be significantly activated in AD and DS. Our results indicate that temporary pharmacological inhibition of the FXII-FVII proteolysis pathway, e.g., using enoxaparin (EP), can enable detection of plasma levels of Aβ1-42 and Aβ1-40 in AD, and this allows development of plasma Aβ1-42 or Aβ1-40 as AD biomarkers for disease detection as well as better assessment of disease progression and response to treatment. Our data indicates that Aβ1-40, which differs from Aβ1-42 by only two amino acids, is also degraded in the blood by the FXII-FVII proteolysis pathway. These results indicate that the FXII-FVII proteolysis pathway may play an important role in AD pathogenesis. However, while Aβ degradation in the plasma by this pathway may be disease-preventive, activation of FXII and other factors in the pathway likely leads to bradykinin liberation from HMWK, complement activation via β-FXIIa and kallikrein, and increased clotting activity, which may promote disease development by impacting vascular physiology, immune response, inflammation and occlusion of microvessels by fibrin clots.

Further, the FXII-FVII proteolysis pathway may have translational implications for developing certain protein therapeutics. Many therapeutic proteins have short plasma half-life, and current approaches are aimed at slowing their removal via biliary, hepatic or renal elimination. Our findings raise the possibility that some of these proteins may engage the FXII-FVII pathway and that inhibiting this pathway may increase their retention in plasma. As a case in point, EP was used as a dose reducer for hPEPD for inhibition of ERBB2-driven tumors in mice.

The present method is based on the identification of the existence of a degradation pathway by which Aβ1-42 is rapidly degraded. The pathway involves the factors in the blood coagulation pathway, although this proteolytic function of the factors appears distinct from the coagulation function. In one embodiment, an agent that inhibits any one or more of the steps of the blood proteolytic pathway (termed herein as a “proteolytic pathway inhibitor”), may be used. In one embodiment, the inhibitor is a low molecular weight heparin (LMWH). These are generally used as anticoagulants. Examples include ardeparin, bemiparin, certoparin, enoxaparin, dalteparin, nadroparin, reviparin, parnaparin and tinzaparin. Other non-LMWH anticoagulants which are inhibitors of the proteolysis pathway including agents targeting FXa and/or FIIa, such as apizaban, rivaroxaban and dadigatran, as well as warfarin and heparin may also be used. Further, antibodies, including monoclonals, polyclonial, hybrid, chimeric, humanized antibodies, nanobodies, and the like and/or antigenic binding fragments thereof can also be used. The antibodies or the peptide mimics may bind to and/or interfere with the function of one or more factors in the cascade including FXII, FIX, FX, FII, and/or FVII. In various embodiments, the LMWH, the non- LMWH small molecule, peptide mimic or antibody interferes with FXII, FIX, FX, and/or FII. It is preferred that the inhibitor does not significantly interfere with normal hemostasis.

We have identified a particular role for FVII. As such, any inhibitor that acts on a step upstream of FVII or that directly affects FVII may be used. In one embodiment, the inhibitor is such that it inhibits the blood proteolytic pathway (also referred to herein as blood proteolysis pathway or extracellular proteolysis pathway) but does not affect, or minimally affects, hemostasis. Examples include specific inhibitors of FXII, such as for example, a small molecule inhibitor or an antibody and the like. The inhibitor can be enoxaparin or a pharmaceutically acceptable salt thereof. For example, the enoxaparin may be sodium enoxaparin. This is available under the trade names Lovenox, Xaparin and Clexane.

The inhibitor may be administered by any means to introduce it into the circulation of the individual. For example, the inhibitor may be introduced via intraperitoneal, intravenous, intramuscular, intradermal, intranasal, subcutaneous, oral, and the like. The inhibitor may also be administered orally in the form of pills, tables, capsules, liquid portions and the like.

The inhibitor or inhibitors can be provided in pharmaceutical compositions for administration by combining them with any suitable pharmaceutically acceptable carriers, excipients and/or stabilizers. Examples of pharmaceutically acceptable carriers, excipients and stabilizer can be found in Remington: The Science and Practice of Pharmacy (2005) 21st Edition, Philadelphia, Pa. Lippincott Williams & Wilkins. For example, suitable carriers include excipients, or stabilizers which are nontoxic to recipients at the dosages and concentrations employed, and include buffers such as acetate, Tris, phosphate, citrate, and other organic acids; antioxidants including ascorbic acid and methionine; preservatives such as octadecyl dimethyl benzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; and/or non-ionic surfactants such as Tween or polyethylene glycol (PEG). The pharmaceutical compositions may comprise other therapeutic agents.

A composition comprising an inhibitor may be administered one or more times. The composition may comprise more than one inhibitor. Multiple administrations can be carried out during the day and this can be continued over a suitable period of time such as from 1-10 days or longer before blood drawing for measurement of Aβ1-40, Aβ1-42 and other Aβ peptides.

The multiple administrations may be done via the same route (such as i.v. route) or may be done using different routes. For example, a first dose may be given via an i.v. route and subsequent doses may be administered via a different route (such as an oral route).

The dose of the inhibitor is such that it inhibits the proteolytic degradation in the blood sufficient to reduce or prevent degradation of Aβ1-42, Aβ1-40 or other Aβ peptides. The inhibitor may be administered once or multiple times. Given the benefit of the present disclosure, one skilled in the art can determine a suitable range and/or optimal dose of desired inhibitors. In one embodiment, the dose per administration of enoxaparin is 0.5 mg/kg, 1.0 mg/kg, 1.5 mg/kg, 2.0 mg/kg or 2.5 mg/kg. In one embodiment, the dose is 40 mg subcutaneously one a day for up to 2 weeks. In one embodiment, the dose for enoxaparin is 0.1 mg/kg to 10 mg/kg (and all values to the tenth decimal point therebetween) administered every 12 or 24 hours (or as desired). Doses and regimens for other inhibitors can be determined by those skilled in the art based on the disclosure herein.

Administration doses of the inhibitor of proteolytic degradation may be such that they can be self-administered by individuals. For example, for subcutaneous administration, the inhibitor may be formulated for administration via self-administrable pens (such as those being used for insulin administration). In one embodiment, an individual may be given a supply of injectable or oral formulations with instructions for intake or administration.

The individual tested by the method of the present disclosure may be individuals who are suspected of having AD or who are at risk of having AD. For example, the individual may be suspected of having or is at risk of having sporadic AD. In individual at risk of developing AD can be identified in clinical practice by one skilled in the art. The risk may be related to family history, DS, a head injury, exposure to causative agents, environmental factors, or any other related circumstances.

In addition, the present method may also be used to test the progression of AD in individuals. Thus, the present method can be carried out at desired intervals to monitor the status or progression of AD. Further, the present method may also be used to monitor the effect of therapeutics on the status or progression of AD in individuals being treated with the therapeutics. Thus, in one embodiment, the method may comprise: administering one or more proteolytic pathway inhibitors to an individual who is being treated with an AD therapeutic, after a suitable regimen of inhibitor administration, collecting a fluid sample from the individual, and determining in the fluid sample (or another sample derived therefrom such as plasma or serum from blood), the level of Aβ1-40 and/or Aβ1-42. The levels of Aβ1-40 and/or Aβ1-42 may be compared to reference standards (established from those who are known not to be affected by AD) or may be compared to reference level from the same individual (such as a level before the onset of the therapeutic treatment). In one embodiment, to monitor the status or progression of AD, the method comprises: i) administering at desired intervals of time, one or more proteolytic pathway inhibitors to an individual, ii) after a suitable regimen of inhibitor administration, collecting a fluid sample from the individual, and iii) determining in the fluid sample (or another sample derived therefrom—such as plasma or serum from blood), the level of Aβ1-40 and/or Aβ1-42. The levels of Aβ1-40 and/or Aβ1-42 may be compared to reference standards (established from those who are known not to be affected by AD) or may be compared to reference level from the same individual (such as a level at the first instance of testing the levels of these markers). The levels can be determined over a period of time during which an individual's status is to be monitored or during which time the individual is being treated for the indication. Determination of blood, plasma or serum Aβ peptide levels can be carried out at regular intervals or as clinically indicated.

The biological fluid sample may be blood, plasma, serum, cerebrospinal fluid and the like. The sample may be used fresh or may be stored (refrigerated or frozen) for later use. In the case of blood, plasma may be prepared and used fresh or may be stored for later use. The samples may be collected during the administration regimen of the inhibitor or may be collected after a suitable period after the termination of the administration regimen. For example, a sample may be collected after a few minutes (such as 5 minutes) to several days (such as 10 days) after the last inhibitor dose is administered. Additional samples may be collected as desired.

The presence of amyloid beta fragments may be detected by immunological methods or by analytical chemical methods (such as HPLC or liquid chromatography coupled to mass spectrometry). For example, Aβ1-42 or Aβ1-40 may be detected by ELISA, using commercially available antibodies or assay kits. For example, Aβ1-42 ELISA kit and Aβ1-40 ELISA kit are available from Life Technologies (catalog number: KHB3441 and KHB3481). Antibodies for these antigens are available from Santa Cruz Biotechnologies.

The level of an Aβ peptide in the blood collected after administration of the inhibitor can be compared to a reference value. The reference value may be the level of the same Aβ peptide in the same individual prior to administration of the inhibitor or level of the Aβ peptide in a control population (such as a population of individual or individuals who are not to be afflicted by AD). Alternatively, comparison to a reference value can be made in the form of normalizing the change in the Aβ peptide value against a change in a control protein whose level in blood is known not to be affected by AD. For example, PEPD can be used as a control protein.

In one embodiment, the method further comprises exposing the collected biological fluid to a proteolytic pathway inhibitor to further reduce degradation after the fluid sample has been obtained. For example, enoxaparin may be added to freshly prepared plasma (or the plasma may be otherwise exposed to enoxaparin).

The present disclosure also provides kits for practice of the present method. The kits may comprise one or more of: one or more inhibitors of the proteolytic degradation pathway, optionally, inhibitor administration tools such as pen type syringes, optionally instructions for administration, tools for collection of fluid sample (such as blood drawing syringe and the like), chemicals and instructions for determination of Aβ1-40 and/or Aβ1-42 in the biological samples or fluids prepared from the biological samples (such as serum or plasma).

The present disclosure provides a method for increasing levels of circulating proteolytic products of amyloid precursor protein in an individual comprising administering to the individual a composition comprising an inhibitor of an extracellular degradation pathway. The extracellular degradation pathway, such as illustrated in FIG. 12, may occur in blood. The inhibitor can inhibit one or more steps in the degradation pathway shown in FIG. 12. The inhibitor can be a small molecule inhibitor, a peptide or a protein, e.g., a monoclonal antibody binding and neutralizing the activity of a coagulation factor involved in the degradation pathway. For example, an inhibitor may inhibit conversion of FXII to FXIIa, conversion of FIX to FIXa, conversion of FX to FXa, conversion of FII to FIIa, conversion of FXI to FXIa and conversion of FVII to FVIIa. By “inhibition” of a step is meant that the efficiency of the step (i.e., the generation of the end product of the step) is reduced or the step may be completely blocked.

The present disclosure provides a method for increasing levels of circulating protein, polypeptides or peptides in an individual comprising administering to the individual a composition comprising an inhibitor of an extracellular degradation pathway, such as illustrated in FIG. 12. The protein, polypeptide or peptide may be a therapeutic or diagnostic agent.

Inhibition of the degradation pathway in the blood can be carried out in vivo or in vitro. For example, circulating blood can be exposed to the inhibitor prior to collecting a blood sample from an individual or after collection of the blood sample. The inhibitor may be added immediately to the tube in which blood has been collected or the tube may be exposed to the inhibitor prior to collection of blood.

For example, the present method can comprise: obtaining a sample of blood from an individual, exposing the collected blood (or plasma or serum obtained therefrom) to one or more inhibitors of the extracellular protein degradation pathway (such as, as illustrated in FIG. 12), and determining the levels of desired proteins, polypeptides or peptides. The proteins or polypeptides, or peptides may be proteolytic products of other larger proteins or polypeptides. For example, the polypeptides may be proteolytic products of amyloid beta precursor protein. The proteolytic products can be Aβ1-40 or Aβ1-42.

The following examples are provided to illustrate the present disclosure.

A method for detecting in an individual proteolytic products of amyloid precursor protein in blood comprising: a) administering to the individual an inhibitor of an extracellular proteolytic degradation pathway in an amount sufficient to inhibit one or more steps of the pathway; b) collecting a blood sample from the individual after a suitable period of time following administration of the inhibitor or during the course of administration; and c) determining the amount of Aβ1-40 and/or Aβ1-42 in the sample or a fraction thereof. The inhibitor of the extracellular proteolytic degradation pathway may be a low molecular weight heparin. Examples of inhibitors of extracellular proteolytic pathway include ardeparin, bemiparin, enoxaparin, certoparin, dalteparin, nadroparin, reviparin, parnaparin, tinzaparin, apizaban, rivaroxaban and dadigatran, warfarin and heparin as well as monoclonal antibody or peptide mimetic inhibitor. Based on the disclosure provided herein, other inhibitors can be identified. Any of the inhibitors or their pharmaceutically acceptable salts can be used. Determination of Aβ1-40 and/or Aβ1-42 or other Aβ peptides can be carried out in whole blood, plasma or serum or any other fraction. The inhibitor can be administered one or more times and blood can be collected after a selected or desired period of time. The level of Aβ peptides can be compared to a reference value. The reference may be a value for the evaluated polypeptides or proteins from an individual known not to have the indication or it may be an averaged value over a population of individuals not to have the indication, or it may be a value obtained from the same individual prior to administration of the inhibitor. The individual may be an individual who is diagnosed with, or has, or is at risk of developing Alzheimer's disease.

A method for identifying increased amyloid beta plaques in an individual comprising the steps of: a) administering to the individual an inhibitor of an extracellular proteolytic degradation pathway in an amount sufficient to inhibit one or more steps of the pathway; b) after a suitable period of time during the course of administration or after termination of the administration regimen, collecting a blood sample from the individual; and c) determining the amount of Aβ1-40 and/or Aβ1-42 in the sample; wherein an increase in the amount of Aβ1-40 and/or Aβ1-42 in the sample from the individual compared to a reference sample is indicative of an increase in amyloid beta plaques in an individual. The reference may be a value for the evaluated polypeptides or proteins from an individual known not to have the indication or it may be an averaged value over a population of individuals, or it may be a value obtained from the same individual prior to administration of the inhibitor. The inhibitor of the extracellular proteolytic degradation pathway may be a low molecular weight heparin. Examples of inhibitors of extracellular proteolytic pathway include ardeparin, bemiparin, enoxaparin, certoparin, dalteparin, nadroparin, reviparin, parnaparin, tinzaparin, apizaban, rivaroxaban and dadigatran, warfarin and heparin. Any of the inhibitors or their pharmaceutically acceptable salts can be used. Determination of Aβ1-40 and/or Aβ1-42 or other proteolytic products of APP can be carried out in whole blood, plasma or serum or any other fraction. The inhibitor can be administered one or more times and blood can be collected after a selected or desired period of time. The individual may be an individual who is diagnosed with, or has, or is at risk of developing Alzheimer's disease. The increased amyloid beta plaques may be in the brain.

A method for monitoring the effect of an AD treatment in an individual comprising the steps of: a) administering to an individual, who is being treated for AD, an inhibitor of an extracellular proteolytic degradation pathway in an amount sufficient to inhibit one or more steps of the pathway; b) after a suitable period of time during the course of administration of the inhibitor or after termination of the administration of the inhibitor, collecting a blood sample from the individual; c) determining the level of Aβ1-40 and/or Aβ1-42 in the sample, or plasma or serum prepared from the sample; and optionally d) comparing the level of Aβ1-40 and/or Aβ1-42 in the sample with a reference level to identify of the effectiveness of the AD treatment. The reference may be a value for the evaluated polypeptides or proteins from an individual known not to have the indication or it may be an averaged value over a population of individuals, or it may be a value obtained from the same individual prior to administration of the inhibitor. The blood can be collected following a desired amount of time after start of administration of the inhibitor. The level of Aβ peptides in blood, plasma or serum can be determined prior to start of AD treatment and at desired times following initiation of treatment and/or after cessation or interruption of treatment to provide an assessment of the efficacy of treatment. If necessary, based on the levels of one or more Aβ peptides, the AD treatment can be discontinued, continued or modified.

In one aspect, this disclosure provides kits for detection of blood Aβ peptides. The kit comprises one or more inhibitors of the blood proteolytic pathway and reagents for detection of one or more Aβ peptides. The kit can optionally further comprise buffers, and instructions for administration of the inhibitor and instructions for use of testing reagents. The inhibitor may be an anticoagulant as described herein. In one embodiment, the inhibitor is enoxaparin and the regents are for testing levels of Aβ1-40 and/or Aβ1-42. The inhibitor may be included as multiple containers of individual doses or a combined cumulative dosage. Testing reagents may be for single testing or multiple testing. The testing kits may be for testing of the Aβ peptides by ELISA. Optionally, blood collection devices and containers can also be included.

The following examples are provided to illustrate the invention. They are not intended to be restrictive.

### Example 1

This example describes the identification of a pathway for proteolytic degradation of proteins in the blood. The materials and methods section in this example also applies to Example 2.

### Materials

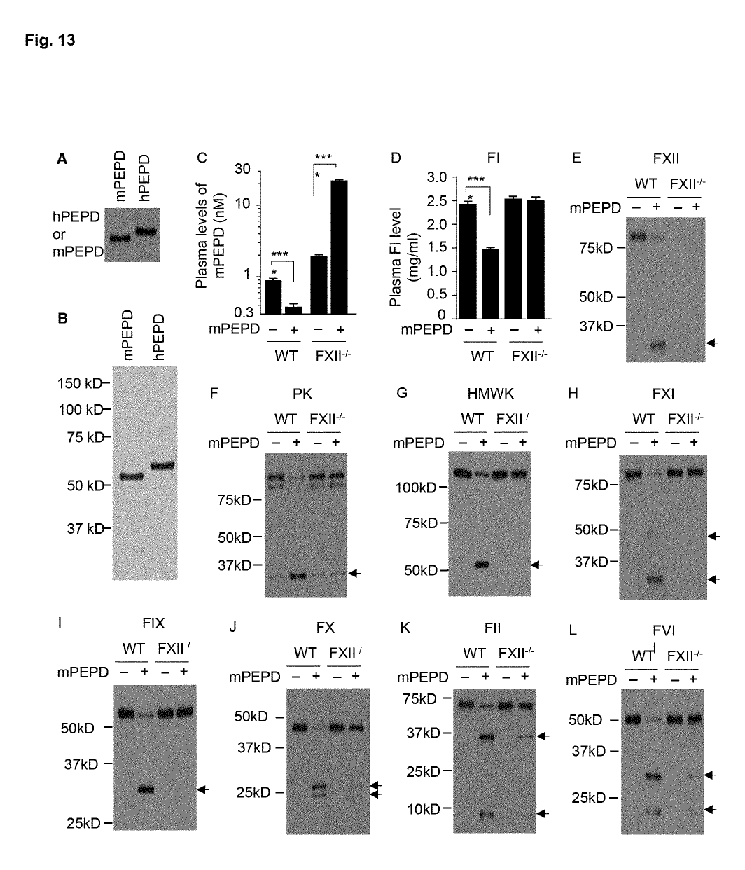

hPEPD) and its mutants (6xHis tagged to the carboxy terminus) were generated, purified and characterized as recently reported (Yang et a., Cell Death Dis 2014; 5: e1211; Yang et al., J Biol Chem 2013; 288(4): 2365-2375). mPEPD was purified from mouse kidney (See Methods; FIGS. 13A and B). hAβ1-42 (A9810) and CCl₄ were purchased from Sigma-Aldrich. mSRC (50311-M20B) and EP were purchased from Sino Biological and Sanofi-Aventis, respectively. The following human coagulation factors were purchased from Haematologic Technologies: FXII (HCXII-0155), FXI (HCXI-0150), FX (HCX-0050), FXa (HCXA-0060), FVII (HCVII-0030), FVIIa (HCVIIA-0031), TF (RTF-0300), FII (HCP-0010), and FIIa (HCT-0020). Human PK (HPK 1302), human HMWK (HK 1300) were purchased from Enzyme Research Laboratories. The following antibodies were purchased from Santa Cruz Biotechnology: Anti-6XHis tag (sc-803), anti-HMWK (sc-25885), anti-FII (sc-16972), anti-SRC (sc-8056), anti-Aβ1-42 (sc-374527, sc-9129), and a donkey anti-goat IgG-horseradish peroxidase (HRP) (sc-2020). The following antibodies were purchased from Cell Signaling Technology: Anti-ERBB1 (2232), anti-pY1173-ERBB1 (4407), anti-ERBB2 (2165), anti-pY1221/1222-ERBB2 (2243), and anti-SRC (2123). The following antibodies were purchased from GeneTex: Anti-FXII (GTX21008), anti-FXI (GTX79765), anti-FIX (GTX79802), anti-FX (GTX110300), and anti-FVII (GTX79785). Anti-PEPD (ab86507) and anti-GAPDH (MAB374) were purchased from Abcam and Millipore, respectively. A donkey anti-rabbit IgG-HRP (NA934V) and a sheep anti-mouse IgG-HRP (NA931V) were purchased from GE Healthcare.

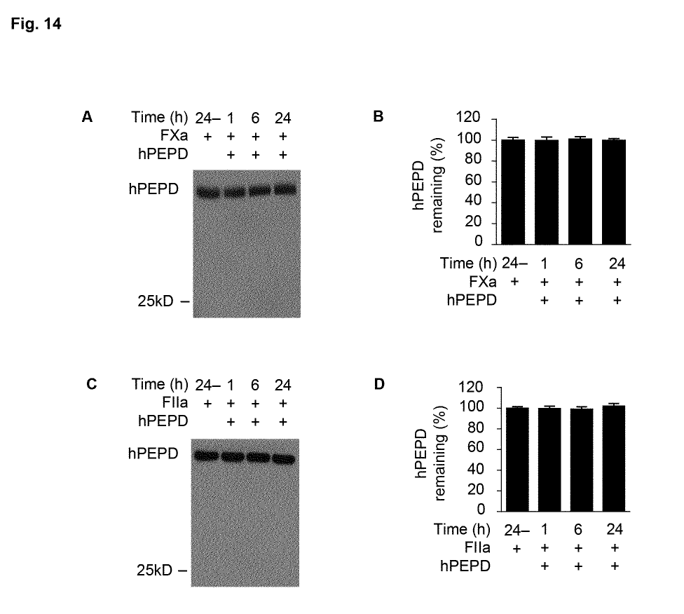

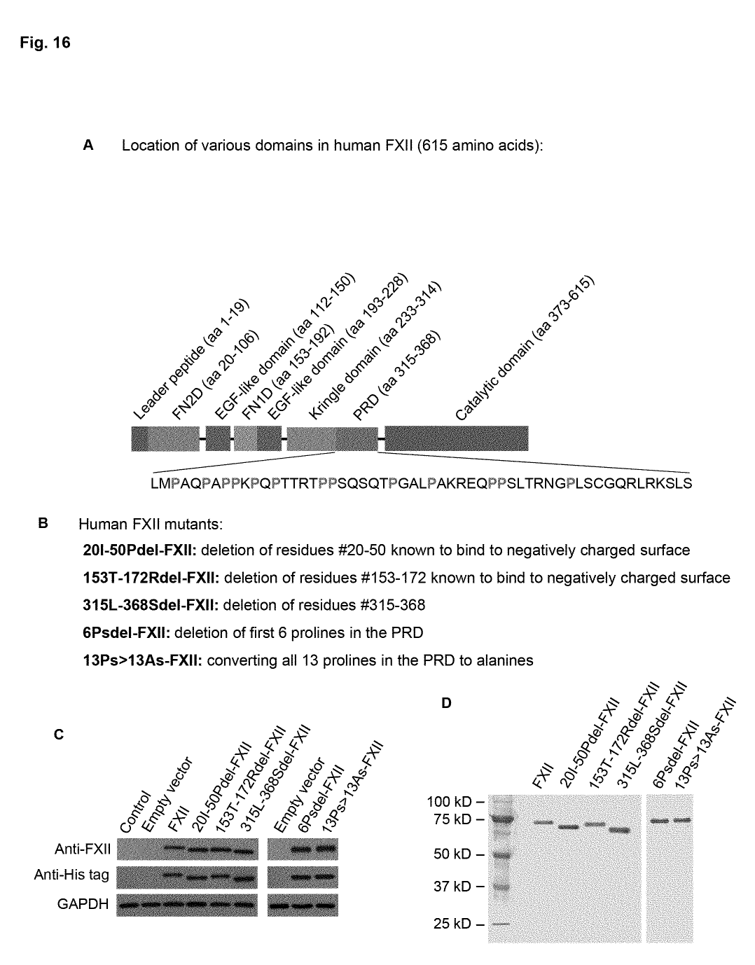

Generation of recombinant Human FXII and its mutants. Recombinant human FXII and its mutants with 6XHis C-terminal tag (FIGS. 14A and B) were generated as described below. The full length human FXII coding sequence without a stop codon was amplified by PCR from normal human liver cDNA using the primers shown in Table 1, which contain unique restriction sites. The PCR conditions were as follows: 95° C. for 3 min, 29 cycles of 94° C. for 30 seconds, 61° C. for 30 seconds, and 68° C. for 2 min, with a final extension at 68° C. for 10 min. Amplified PCR products were digested with applicable restriction enzymes (EcoR1 and SalI), followed by ligation into pCMV6-XL5 (Origene), which was pre-digested with the same restriction enzymes. 6XHis C-terminal tag was added to the expression construct by PCR-based site-directed mutagenesis using the primers listed in Table 1. The insert was confirmed by DNA sequence analysis. Mutation of proline to alanine as well as specific deletion in the FXII coding sequence, including 20I-50Pdel-FXII, 153T-172Rdel-FXII, 315L-368Sdel-FXII, 6Psdel-FXII, and 13Ps>13As-FXII, were accomplished using QuikChange Lightning Multi Site-Directed Mutagenesis Kit or QuikChange Lightning Site-Directed Mutagenesis Kit (Agilent Technologies), using primers listed in Table 1. All the reactions were carried out according to the manufacturer's instructions. All constructs were sequenced to ensure correct changes. These plasmids were used to generate the recombinant proteins in CHO-K1 cells (ATCC). CHO-K1 cells were cultured in F-12K medium (Gibco) supplemented with 10% FBS (Gibco) in a humidified incubator at 37° C. with 5% CO₂. Cells growing in 6-well plates were transfected with the pCMV6-XL5 plasmids expressing FXII or a mutant as described above, using FuGENE HD (Promega), at 1-2 μg of DNA per well for 48 h. Cells were then harvested, washed with PBS and suspended in a lysis buffer (50 mM NaH₂PO₄, 300 mM NaCl, 10 mM imidazole, 0.05% Tween 20, with pH adjusted to 8.0 using NaOH) at 0.5 ml per 10⁷ cells. Cell lysis was enhanced by sonication on ice. The lysates were cleared of debris by centrifugation at 10,000×g for 10 min at 4° C. The 6XHis-tagged FXII and its mutants were purified by Ni-NTA agarose chromatography. The relative molecular size of each protein was checked by IB (FIG. 16C), and high purity of each protein was confirmed by SDS-PAGE and silver staining, using a kit (LC 6070) from Invitrogen (FIG. 16D). Protein concentrations of all samples were measured by the bicinchoninic acid (BCA) protein assay kit (Pierce).

Animal studies. All animal studies were performed in accordance with protocols approved by the Institutional Animal Care and Use Committee at Roswell Park Cancer Institute. Male mice at 7-8 weeks of age were used, including wild type (WT) mice (C57BL/6), and mice deficient in FXII (C57BL/6-FXII^{-/-}) or FVII (C57BL/6-FVII^{tTA/tTA}). WT C57BL/6 mice were purchased from Taconic. C57BL/6-FXII^{-/-} mice and C57BL/6-FVII^{tTA/tTA} mice were bred in our own facility and genotyped. The breeders were kindly provided by Dr. Francis J. Castellino at University of Notre Dame. All treatments were given by i.p. as follows: A single dose of vehicle, hPEPD, mPEPD, mSRC, hAβ1-42 or CCl₄; EP once daily for 5 days, followed at 1 h after the last dose of EP with a single dose of vehicle, hPEPD or hAβ1-42. CCl₄ was dissolved in corn oil, whereas all other substances were dissolved in PBS. The vehicle or the test substance was given to mice in 0.1 ml volume per 20 g body weight. Blood was collected from the mice at specific times by cardiac puncture at the time of sacrifice by carbon dioxide, and heart, liver and kidney were also collected from some of the mice. Blood was collected into K3 EDTA-containing tubes (Multivette 600 from Sarstedt), unless specified otherwise. All blood samples were promptly centrifuged to obtain plasma samples.

Measurement of plasma levels of PEPD, SRC, Aβ1-42 and FI. Plasma concentrations of hPEPD, mPEPD, mSRC, hAβ1-42, mAβ1-42 and FI (also known as fibrinogen) were determined by ELISA. To measure plasma levels of mAβ1-42, total Aβ1-42 (mAβ1-42 plus hAβ1-42) or mSRC, 96-well ELISA plates were coated with an anti-Aβ1-42 mouse monoclonal antibody (sc-374527) or an anti-SRC mouse monoclonal antibody (sc-8056) at 0.25 μg/100 μl/well at 4° C. overnight. The plates were washed three times with phosphate buffered saline tween-20 (PBST) and the coated wells were blocked by incubation with 200 μl/well of 1% BSA in PBS for at least 2 h at RT. After another round of wash with PBST, the plates were incubated with appropriately diluted Aβ1-42 standard, SRC standard or plasma samples (100 μl/well) for 2 h at RT. The plates were then washed with PBST and incubated with an anti-Aβ1-42 rabbit polyclonal antibody (sc-9129) or anti-SRC rabbit monoclonal antibody (2123) at 100 μl/well for 2 h at RT. The plates were washed again with PBST, and each well was incubated with a goat anti-rabbit IgG-HRP conjugate (100 μl) for 1 h at RT. After yet another round of wash with PBST, each well was incubated with 100 μl of HRP substrate 3,3′,5,5′-tetramethylbenzedine (Cell Signaling, 7004). Upon adequate color development, 100 μl of stop solution (Cell Signaling, 7002) was added to each well, and absorbance at 450 nm was promptly recorded by a microtiter plate reader. Plasma FI concentration was determined using an assay kit (400-374-130050) from GenWay Biotech, following the manufacturer's instruction.

IB. Tissue samples were mixed with RIPA buffer (25 mM Tris-HCl, pH 7.6, 150 mM NaCl, 1% Nonidet P-40, 1% sodium deoxycholate, 0.1% SDS), supplemented with 2 mM phenylmethanesulfonyl fluoride, a proteinase inhibitor mix (Roche Applied Science) and phosphatase inhibitor Cocktail 2 (Sigma-Aldrich). Tissue samples were stroked in a Dounce homogenizer, and the homogenates were cleared by centrifugation at 12,000×g for 15 min at 4° C. Protein concentrations in all samples were measured by the BCA assay kit. Plasma samples were used without further processing (20 μl per sample). Each sample was mixed with 4× loading dye, heated for 5 min at 95° C. and then resolved by SDS-PAGE (8-12.5%). Notably, 7.5 μl of original plasma per sample was analyzed. The proteins were transferred to polyvinylidene fluoride membrane, probed with specific antibodies and detected using the ECL Plus Kit (Amersham) or the SuperSignal West Pico Kit (Thermo Scientific).

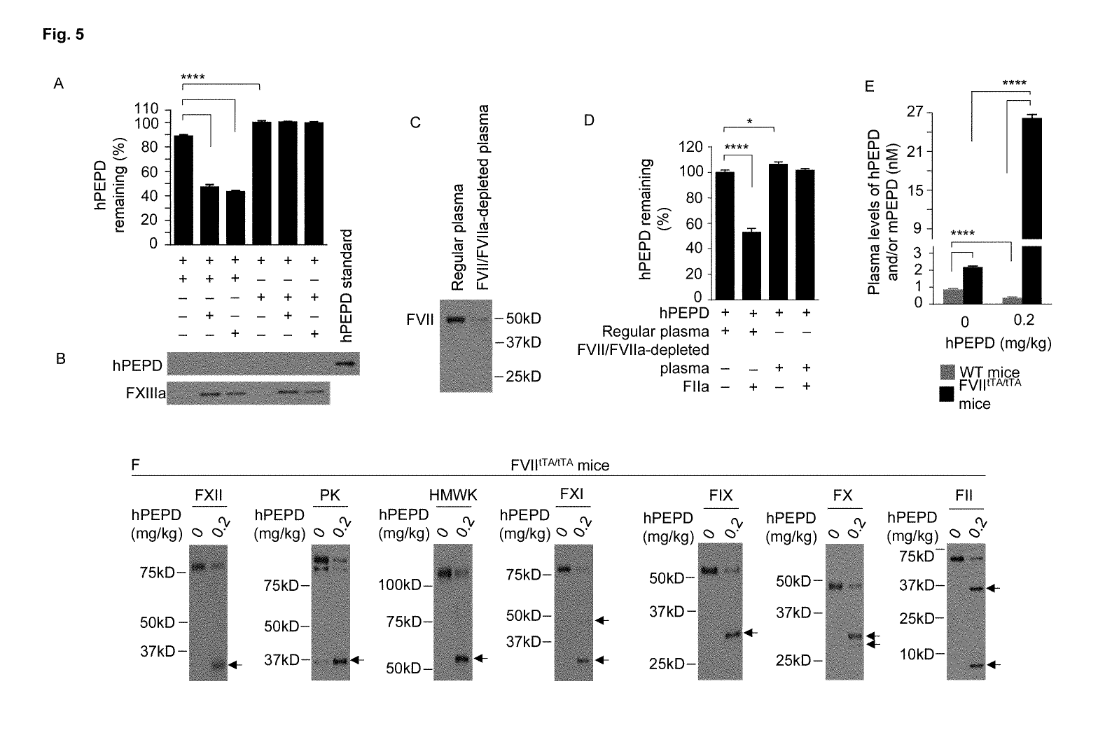

Measurement of in vitro degradation of hPEPD, mSRC and hAβ1-42. To determine whether a blood coagulation factor degrades hPEPD, hPEPD at 90 nM or lower concentrations (10 or 40 nM) was incubated with solvent, 100 nM FIIa, 100 nM FXa, 10 nM FVIIa with or without 10 nM TF, or 10 nM FVII with or without 10 nM TF and/or 100 nM FIIa in PBS in a total volume of 100 μl containing 5 mM CaCl₂ for desired times at RT. Notably, TF was solubilized in 10 mM CHAPS, which was diluted by 10-fold in the final assay. To compare the hPEPD-degrading activities of different plasma samples, 9.2 pmol of hPEPD was incubated with 100 μl of plasma from WT mice or FVII^{tTA/tTA} mice at RT, with or without FIIa or FXa (10 pmol), and to maximize detection of any activity, all incubations lasted for 24 h. Notably, blood was drawn into plastic tubes without an anticoagulant but immediately centrifuged to remove cells and platelets; we refer to such sample as plasma in this paper. Although some clotting activity may take place before and during centrifugation of the blood sample, our experiments indicate that activation of coagulation factors, e.g., FVII, is very limited (see FIG. 5A). To measure remaining hPEPD, the incubated samples were cleared of potential fibrin clots by centrifugation and then analyzed by SDS-PAGE and silver staining (using the LC 6070 kit from Invitrogen), IB or measurement of hPEPD enzymatic activity. hPEPD enzymatic activity was measured using glycyl-proline as a substrate (as described in Zbucka et al., Folia Histochem Cytobiol 2007; 45 Suppl 1: S181-185). The precipitates from each incubated sample were washed with PBS twice and then resuspended in 2.5% SDS (20 μper sample); each solution was mixed with 10 μl loading dye and analyzed for hPEPD by IB, using pure hPEPD as a standard and factor FXIIIa as a binding control, respectively.

To measure degradation of hPEPD in plasma samples with or without removal of FVII and FVIIa, plasma samples from WT mice as described above were either used directly or immunodepleted of FVII and FVIIa before use. To remove FVII and FVIIa from plasma, 500 μl plasma was incubated with an antibody specific for FVII/FVIIa (GTX79785; 10 μg) and protein A sepharose beads for 1 h at RT. Following centrifugation at 13,000×g for 1 min at 4° C., the supernatant fraction was collected and analyzed by IB to confirm depletion of FVII and FVIIa. Next, 9.2 pmol of hPEPD was incubated with 100 μl of regular plasma or the plasma depleted of FVII/FVIIa, with or without FIIa (10 pmol) for 24 h at RT. Potential fibrin clots formed during the incubation was removed by centrifugation at the end of the incubation, and remaining hPEPD was measured by hPEPD enzymatic activity analysis.

To measure degradation of mSRC or hAβ1-42 by FVIIa, mSRC (0.17 μM) or hAβ1-42 (2.2 μM) was incubated with or without FVIIa (10 nM) and TF (10 nM) in PBS in a total volume of 100 μl containing 5 mM CaCl₂ for desired times at RT. All samples after incubation were resolved by SDS-PAGE and stained by silver. In the case of Aβ1-42 which aggregated during the incubation, the aggregates were re-dissolved in 30 μl of 2% SDS and combined with the supernatant fraction before SDS-PAGE.

Measurement of binding of hPEPD, mSRC or hAβ1-42 to a coagulation factor or its mutant. Binding reactions were carried out in PBS in a total volume of 100 μl for 2 h at 37° C. To assess binding of hPEPD to HMWK, PK, FXI, FXII or a FXII mutant, hPEPD at 40 nM was incubated with a potential binding partner at 0.5 μM. All FXII mutants are His tagged, while FXII is either 6XHis-tagged (generated in our own laboratory) or not His-tagged (purchased commercially), depending on the experiment. To compare hPEPD and its mutants for binding to FXII, hPEPD or a mutant (40 nM) was incubated with FXII at 0.5 μM. Likewise, to assess binding of mSRC to FXII and its mutants, mSRC (40 nM) was incubated with FXII or a mutant at 0.5 μM. At the end of the incubation, 300 μl PBS containing a specific primary antibody was added to the incubation solution, which was further incubated at 4° C. overnight, followed by IP by protein G-agarose, and the precipitates were analyzed by IB. The above approach was not used to assess binding of hAβ1-42 to FXII and its mutants, due to the tendency of hAβ1-42 to aggregate. Instead, hAβ1-42 (200 nM) was incubated with FXII or a mutant (20 nM) in PBS in a total volume of 100 μl for 2 h at RT; the solution was then centrifuged at low speed to separate the supernatants from the precipitates. The precipitates were re-dissolved in 30 μl of 2% SDS, which were analyzed along with the supernatants by IB.

Measurement of FXII activation. FXII activation by hPEPD, its mutants, mSRC or hAβ1-42 in vitro was measured by a chromogenic assay as described in previously (Maas et al., J Clin Invest 2008; 118(9): 3208-3218). hPEPD and its mutants were each evaluated at 40 and 400 nM. hAβ1-42 was evaluated at 0.05, 0.5 and 11.1 μM. mSRC was evaluated at 20 and 200 nM. FXII activation was followed for 3 h at RT by monitoring the conversion of the chromogenic substrate at 405 nm by a microtiter plate reader. Notably, the assay specifically measures FXII activation, as omission of FXII from the reaction solution abrogated conversion of the chromogenic substrate in all reactions in our experiments.

Statistical analysis. Data were analyzed by analysis of variance (ANOVA), followed by Tukey multiple comparisons test. For data that are highly skewed, log transformation was performed before ANOVA, as indicated in figure legend. P value of 0.05 or lower was considered statistically significant.

### Additional Materials and Methods

Materials. Human coagulation factors FX (HCX-0050) and FII (HCP-0010) were purchased from Haematologic Technologies.

Preparation of Mouse PEPD (mPEPD). mPEPD was obtained from the kidneys of C57BL/6 mice by immunoaffinity purification. First, a PEPD antibody was covalently linked to protein A-sepharose beads. Five mg of anti-PEPD (Ab111851, Abcam) was incubated with 50 ml of 6 mg/ml protein A-sepharose beads (17-6002-35, GE Healthcare Life Sciences) at RT for 1 h. The antibody-bound beads were washed three times with PBS and incubated with cross linker B S3 (Pierce) at 2 mM for 30 min (RT). The cross-linking reaction was terminated by adding 50 mM Tris to the mixture (final, pH 7.5), followed by incubation at RT for 15 min. The antibody-bound beads were washed three times with PBS, and the beads from each cross-linking reaction was incubated in 1 ml of blocking buffer containing 100 mM ethanolamine (pH 8.2) at RT for 15 min, in order to block any remaining NHS-ester groups on BS3, which was followed by washing the bead complexes with PBS three times. Next, kidneys from 8-week-old C57BL/6 mice were minced with scissors and homogenized to 10 times the volume (v/w) in ice-cold 50 mM Tris-HCl (pH 7.4) with a Dounce homogenizer. The homogenates were centrifuged at 9000×g for 20 min (4° C.) to remove tissue debris. The supernatant sample was incubated with the PEPD antibody-protein A-sepharose bead conjugates (0.5 ml sample with 50 ml beads) overnight at 4° C. The beads were then washed with 50 mM Tris (pH 8.0) three times, and the bound mPEPD molecules were eluted with 1 ml of 100 mM glycine-HCl (pH 2.8) four times. The eluates were pooled, and the pH of the solution was adjusted to approximately 7.4, using 1 M Tris-HCl (pH 9.5). The mPEPD solution was then concentrated using Ultracel YM-30 Centricon (Millipore). Protein concentration in the sample was measured by the BCA protein assay kit. The mPEPD preparation was checked by western blotting, and its purity was confirmed by SDS-PAGE, followed by silver staining (FIGS. 13A and B), using hPEPD for comparison.

Measurement of Potential Activation of FX, FII or FVII by hPEPD. hPEPD (40 nM) was incubated with FX, FIT or FVII (0.5 mM) in PBS at RT in the presence of 5 mM CaCl₂ for 24 h, followed by IB analysis for potential activation/cleavage of each coagulation factor. FXa, FIIa and FVIIa were used as positive controls in the experiments.

Measurement of Plasma Levels of Aspartate Transaminase (AST) and Alanine Transaminase (ALT). Plasma AST activity was measured using the Infinity™ AST (GOT) Reagent kit (Thermo, TR70121). Plasma ALT activity was measured using the Infinity™ ALT (GPT) Reagent kit (Thermo, TR71121). Each assay was performed according to the manufacturer's instruction.

### Results

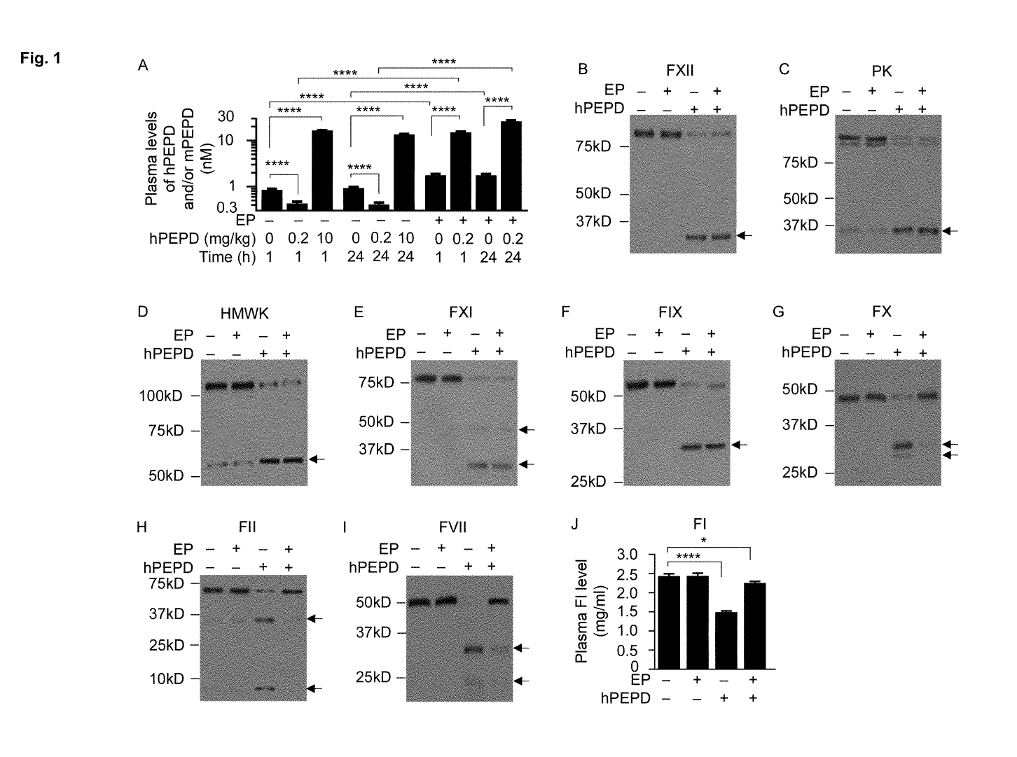

PEPD is degraded in the plasma by coagulation proteases but enoxaparin inhibits the degradation. Average plasma level of mouse PEPD (mPEPD) was 0.9 nM in control mice, which is very similar to our previously obtained values (Yang et al., J Biol Chem 2013; 288(4): 2365-2375), and average plasma levels of total PEPD (hPEPD plus mPEPD) increased 19.4- and 15.1-fold at 1 and 24 h, respectively, following intraperitoneal injection (i.p.) of recombinant human PEPD (hPEPD) at 10 mg/kg (FIG. 1A). Surprisingly, at 1 and 24 h following hPEPD injection at 0.2 mg/kg, average plasma levels of total PEPD were only 51% and 46% of control, respectively (FIG. 1A). This suggested that hPEPD might elicit rapid elimination of itself and mPEPD from plasma in mice.

Activators of antithrombin seem to elevate plasma PEPD level in rats. We treated mice with enoxaparin (EP), a clinically used low molecular weight heparin which activates antithrombin III, at 2.5 mg/kg i.p. daily for 5 days and gave hPEPD (0.2 mg/kg) i.p. at 1 h after the last EP dose; average plasma levels of total PEPD were 35.2- and 63.8-fold higher at 1 and 24 h, respectively, following hPEPD treatment than in mice treated with the same dose of hPEPD alone (FIG. 1A). EP also increased average plasma level of mPEPD by 2.1-fold (FIG. 1A). hPEPD treatment caused cleavage or activation of many coagulation factors in mice, including factor XII (FXII), prekallikrein (PK), high molecular weight kininogen (HMWK), factor XI (FXI) factor IX (FXI), factor X (FX), factor II (FII), factor I (FI) and factor VII (FVII) (FIG. 1B-J). FXII, PK, HMWK, FXI, FIX are components of the intrinsic blood coagulation cascade, whereas FVII belongs to the extrinsic blood coagulation cascade, which is typically activated via tissue factor (TF) upon blood vessel damage. The two cascades converge on FX, activation of which leads to FII activation, which cleaves FI. EP itself had no effect on these factors and failed to prevent hPEPD from activating FXII, PK, HMWK, FXI and FIX, but prevented hPEPD from causing activation or cleavage of FX, FII, FI and FVII (FIG. 1B-J). EP is known to inhibit FIXa, FXa and FIIa (the active forms of FIX, FX and FII) by activating antithrombin III. Because both FIIa and FXa can activate FVII, it suggested that hPEPD activated FVII by activating the intrinsic and common coagulation cascades. Although hPEPD is of human origin and has a His tag, injecting mPEPD without any tag to mice elicited the same response as did hPEPD (FIG. 13 C-L).

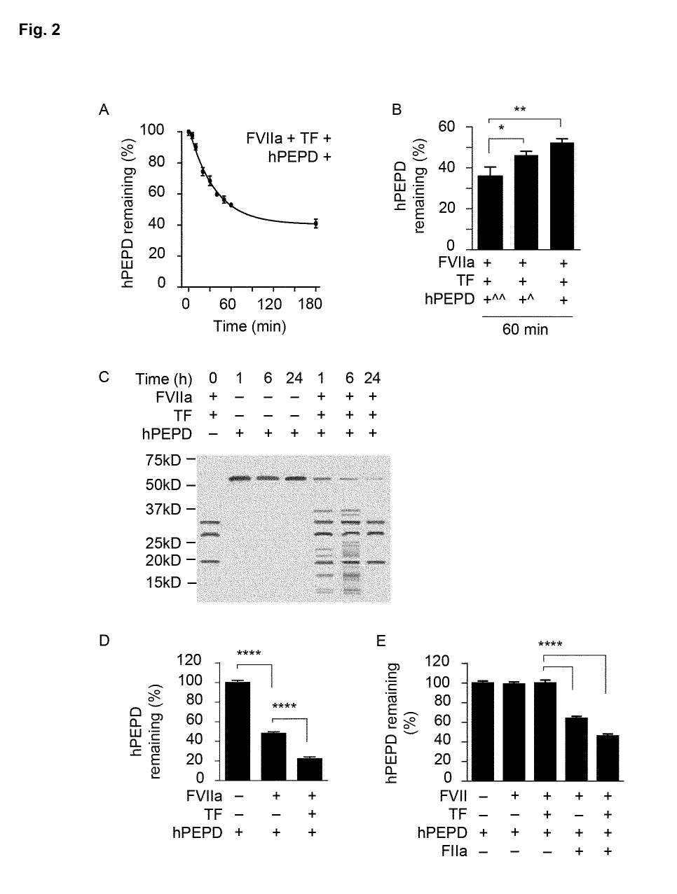

We next evaluated the effects of several human coagulation factors on hPEPD. We focused on FXa, FIIa and FVIIa (active form of FVII), as EP inhibition of PEPD degradation was associated with inhibition of these factors. FXa and FIIa had no effect on hPEPD stability (FIG. 14A-D), but FVIIa (10 nM) plus TF (10 nM) caused rapid and extensive hPEPD proteolysis (FIG. 2A-C). For example, only 35.9%, 46.3% and 52.4% of hPEPD remained after it was incubated at 10, 40 and 90 nM with FVIIa and TF for 60 min, respectively (FIG. 2B). Notably, given that the normal plasma concentration of FVII is approximately 10 nM (Bajaj et al., J Biol Chem 1981; 256(1): 253-259), the FVIIa concentration used in the above experiment is probably much higher than its physiological concentration, which was intended to facilitate detection of its activity. However, as described later, FVIIa is essential for hPEPD degradation in the plasma. Although TF enhances the proteolytic activity of FVIIa, as expected, it may not necessarily be involved in PEPD degradation by FVIIa in vivo, as TF is present in subendothelial tissue, and no blood vessel injury appears to be involved in the activation of FVII and other coagulation proteases in mice injected with hPEPD. While a soluble bioactive form of TF circulates in blood, potential involvement of this factor in PEPD degradation by FVIIa remains unknown. Notably, FVIIa in the absence of TF degraded hPEPD (FIG. 2D). Moreover, while FVII with or without TF showed no proteolytic activity towards hPEPD, adding FIIa at a physiologically relevant concentration (100 nM) to the mixture caused marked hPEPD degradation (FIG. 2E). These results further show that hPEPD activates FVII via the intrinsic and common coagulation cascades and also reveal that FVIIa degrades hPEPD. FVIIa is a trypsin-like serine protease, cleaving peptide bonds at the carboxyl side of arginine and lysine and is known to cut FIX and FX. The extensive proteolysis of hPEPD by FVIIa, however, contrasts with its limited proteolysis of FIX (two proteolytic sites) and FX (one proteolytic site) (Schmidt et al., Trends Cardiovasc Med 2003; 13(1): 39-45; Leonardi et al., Toxicon 2008; 52(5): 628-637). The proteolytic sites in hPEPD for FVIIa are not yet known.

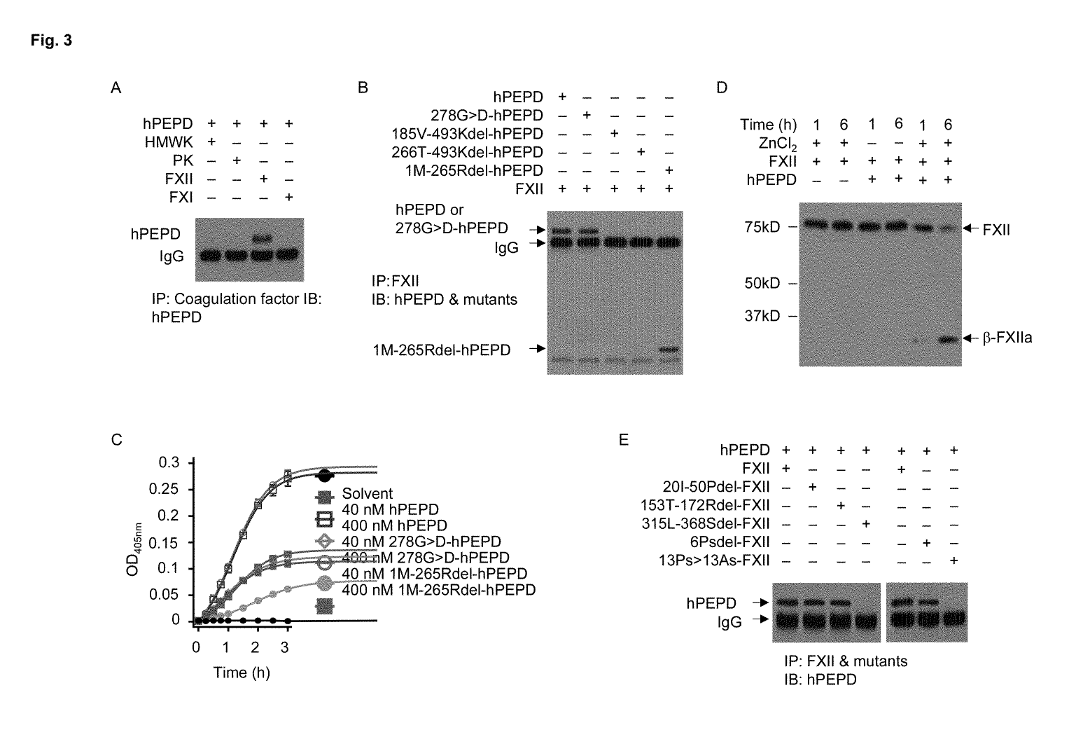

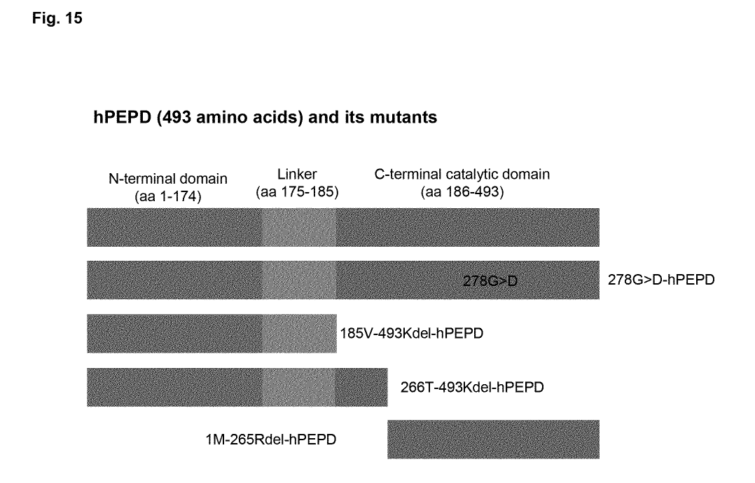

hPEPD directly binds and activates FXII. We next evaluated the interaction of hPEPD with several human coagulation factors. FXII interacts with HMWK, PK and FXI to initiate the intrinsic coagulation cascade, but hPEPD binds only to FXII (FIG. 3A). hPEPD is a homodimeric protein, with each subunit composed of 493 amino acids, containing the N-terminal regulatory domain, a linker and the C-terminal catalytic domain (amino acids #1-174, 175-185 and 186-493, respectively). We evaluated the interaction of FXII with four hPEPD mutants, including 278G>D-hPEPD, 1M-265Rdel-hPEPD, 185V-493Kdel-hPEPD and 266T-493Kdel-hPEPD (FIG. 15). The mutants, except 278G>D-hPEPD, cannot form homodimers (Yang et al., Cell Death Dis 2014; 5: e1211). Enzymatically inactive 278G>D-hPEPD (Ledoux et al., Am J Hum Genet 1996; 59(5): 1035-1039) and 1M-265Rdel-hPEPD were almost indistinguishable from hPEPD in FXII binding, while neither 185V-493Kdel-hPEPD nor 266T-493Kdel-hPEPD could bind to FXII (FIG. 3B). FXII is a single chain zymogen; proteolytic cleavage at R353-V354 generates FXIIa, with the N-terminal heavy chain and C-terminal catalytic light chain ((3-FXIIa) held together by a disulfide bond (Stavrou et al., Thromb Res 2010; 125(3): 210-215). hPEPD and 278G>D-hPEPD did not differ in activating FXII, whereas 1M-228Hdel-hPEPD was about 50% active (FIG. 3C). These results show: 1) the enzymatic function of hPEPD is not involved in FXII binding and activation; 2) each hPEPD monomer binds to FXII independently; 3) the C-terminal sequence of hPEPD binds to FXII, but additional sequence in hPEPD is involved in full XII activation. Zn²⁺ is involved in FXII activation. FXII activation by hPEPD also required Zn²⁺ (FIG. 3D).

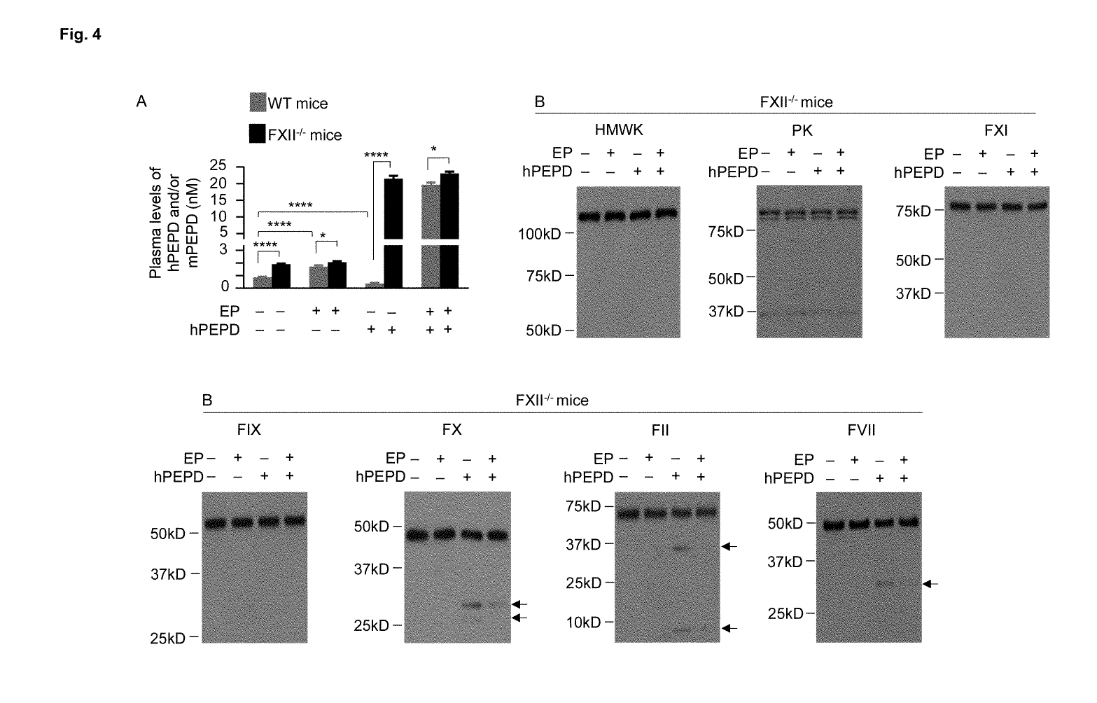

We next compared human FXII with its mutants for binding to hPEPD, including 20I-50Pdel-FXII, 153T-172Rdel-FXII, 315L-368Sdel-FXII, 6Psdel-FXII, and 13Ps>13As-FXII (FIG. 16A-4D). While negatively charged surfaces and other substances activate FXII by binding to sequences within the fibronectin type II domain (FN2D) or fibronectin type I domain (FN1D) in FXII, FXII mutants lacking the relevant binding sites (20I-50Pdel-FXII and 153T-172Rdel-FXII) bound to hPEPD as well as did FXII (FIG. 3E). However, the FXII mutant lacking the proline-rich domain (PRD; 315L-368Sdel-FXII) could not bind to hPEPD (FIG. 3E). Of the 13 prolines in the PRD of FXII (FIG. 16A), deletion of the first 6 prolines (6Psdel-FXII) only slightly attenuated FXII binding to hPEPD, whereas replacing all 13 prolines with 13 alanines (13Ps>13As-FXII) completely abolished its binding to hPEPD (FIG. 3E). These results show that hPEPD binds to FXII via PRD and that most if not all of the prolines are involved in the binding. This also reveals a physiological function of PRD in FXII.

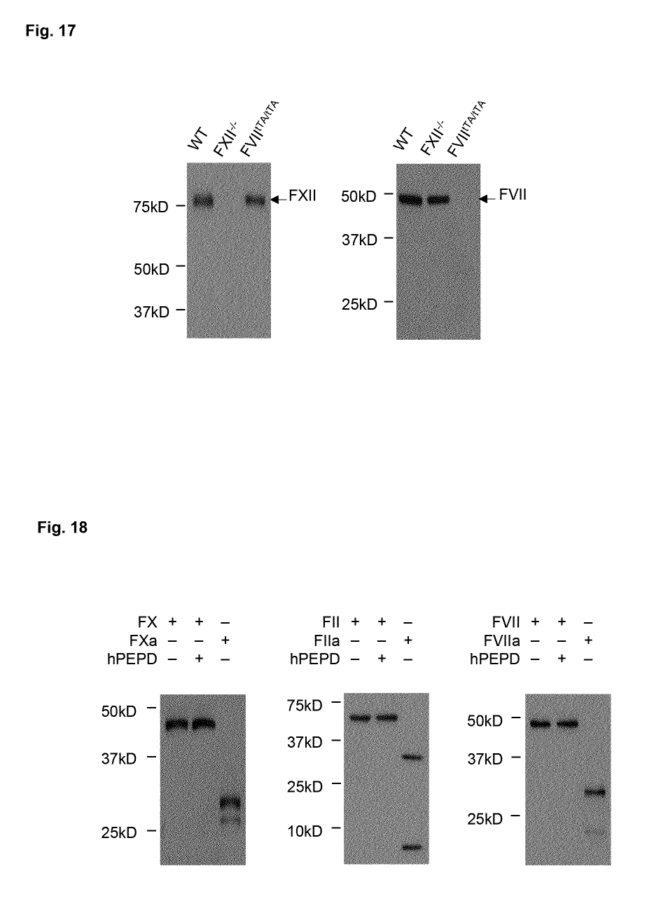

FXII initiates PEPD degradation in the plasma. We further studied plasma PEPD degradation using FXII knockout mice (C57BL/6-FXII^{-/-}), their WT counterparts, and EP. FXII was absent in the plasma of FXII^{-/-} mice (FIG. 17), in which exons 3-8 of the FXII gene is replaced with the neomycin resistance gene. Basal plasma level of mPEPD was 2.2-fold higher in FXII^{-/-} mice than in WT mice (FIG. 4A). EP treatment (2.5 mg/kg i.p. daily for 5 days) elevated plasma level of mPEPD by 2.0-fold in WT mice but not in FXII^{-/-} mice (FIG. 4A). Without EP pretreatment, at 6 h following hPEPD treatment (0.2 mg/kg i.p.), plasma level of total PEPD decreased 66% in WT mice, as shown before, but increased 12.5-fold in FXII^{-/-} mice (FIG. 4A); average plasma level of total PEPD is 53.8-fold higher in FXII^{-/-} mice than in WT mice. With EP pretreatment, at 6 h following hPEPD treatment (0.2 mg/kg i.p.), average plasma level of total PEPD was 19.7 nM in WT mice, which is still 14.6% lower than in FXII^{-/-} mice (FIG. 4A), indicating that EP did not fully inhibit FXII-initiated PEPD degradation in WT mice. However, EP did not show a significant effect on plasma PEPD level in FXII^{-/-} mice (FIG. 4A). These results show that FXII is essential for degradation of plasma PEPD. In FXII^{-/-} mice, hPEPD treatment caused no activation of HMWK, FXI, PK and FIX, but slight activation of FX, FII and FVII (FI was not measured), which was largely inhibited by EP (FIG. 4B). The mechanism of FXII-independent activation of FX, FII and FVII by hPEPD remains unknown, but hPEPD does not directly activate any of these factors (FIG. 18). Our results do not show that FXII-independent activation of FVII contributes significantly to plasma PEPD degradation. Lack of EP effect on plasma PEPD level in FXII^{-/-} mice, contrary to its ability to markedly elevate plasma PEPD level in WT mice (FIG. 1A), also further shows that EP elevates plasma PEPD level in WT mice by inhibiting the FXII-mediated proteolysis pathway, rather than by modulating PEPD exit from plasma, e.g., its sequestration in the matrix or its uptake by cells. Collectively, we conclude that plasma PEPD degradation originates from direct FXII activation by PEPD.

Plasma PEPD is degraded exclusively by FVIIa. We next turned to FVII-deficient mice. Replacement of both FVII alleles in mice with a construct containing the tetracycline transactivator (tTA) promoter attached to the FVII cDNA (FVII^{tTA/tTA}) results in negligible FVII expression (Rosen et al., Thromb Haemost 2005; 94(3): 493-497). FVII was undetectable in the plasma of FVII^{tTA/tTA} mice (FIG. 17). We compared the hPEPD-degrading activities of plasma samples from FVII^{tTA/tTA} mice and WT mice. Notably, blood was drawn from mice without an anticoagulant (EDTA), in order to assess the activities of FVII and other factors, but was immediately centrifuged to remove cells and platelets. Under the same experimental condition, no degradation of hPEPD could be detected after incubation with FVII^{tTA/tTA} plasma, but only 88.8% of hPEPD remained after incubation with WT plasma (FIG. 5A), implying the presence of a low level of FVIIa in WT plasma, either preformed or generated during the incubation. Adding human FIIa or FXa to WT plasma markedly enhanced hPEPD degradation, but neither factor was effective in FVII^{tTA/tTA} plasma (FIG. 5A). Notably, all incubated solutions were cleared of precipitates (potential fibrin clots) by centrifugation before analysis. The precipitates after wash with PBS were suspended in 2.5% SDS and analyzed by IB for presence of hPEPD, using factor XIIIa (FXIIIa) as the binding control, since FXIIIa is known to bind to fibrin. However, no hPEPD could be detected in any of the samples (FIG. 5B), although FXIIIa was also absent in two samples, apparently due to loss of the minute amount of precipitates during PBS wash. Moreover, removal of FVII/FVIIa from WT plasma by immunodepletion abolished the residual hPEPD degradation or FIIa-enabled hPEPD degradation (FIGS. 5C and D). Basal plasma level of mPEPD was 2.5-fold higher in FVII^{tTA/tTA} mice than in WT mice (FIG. 5E). At 6 h following hPEPD treatment (0.2 mg/kg i.p.), plasma level of total PEPD decreased 56.3% in WT mice but increased 12.0-fold in FVII^{tTA/tTA} mice, differing by 68.9-fold between the two genotypes (FIG. 5E). FVII^{tTA/tTA} mice closely resemble FXII^{-/-} mice with regard to their inability to degrade plasma PEPD, but the coagulation factors that were not activated in hPEPD-treated FXII^{-/-} mice, as shown in FIG. 4B, were all activated in hPEPD-treated FVII^{tTA/tTA} mice, excluding FVII (FIG. 5F). Collectively, we conclude that plasma PEPD is degraded exclusively by FVIIa and that both FXa and FIIa, formed after FXII activation by PEPD, activate FVII.

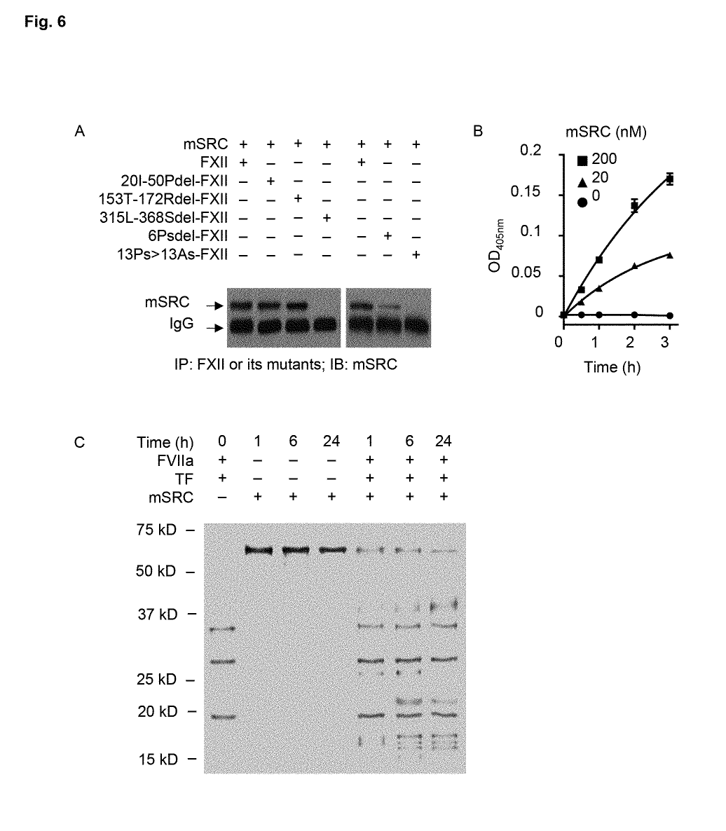

Degradation of plasma SRC by the FXII-FVII proteolysis pathway. Many proteins bind to proline-rich motifs via SH3, WW or EVH1 domains. We examined the interaction of SH3-containing mouse SRC (mSRC) with the FXII-FVII pathway. Although mSRC is an intracellular tyrosine kinase, it is present in plasma at a low concentration as shown later, probably due to release from damaged cells. mSRC directly bound and activated FXII (FIGS. 6A and B). Deletion of the PRD in FXII (315L-368Sde1-FXII) completely abolished mSRC binding, but binding was not altered by deletion of the sequences in FN2D or FN1D that binds to negatively charged surfaces and other substances (20I-50Pdel-FXII or 153T-172Rdel-FXII) (FIG. 6A). However, mSRC differs from hPEPD in that its binding to FXII was severely reduced after deletion of the first 6 prolines in the PRD of FXII (FIG. 6A), whereas such deletion only slightly attenuated hPEPD binding to FXII as described before.

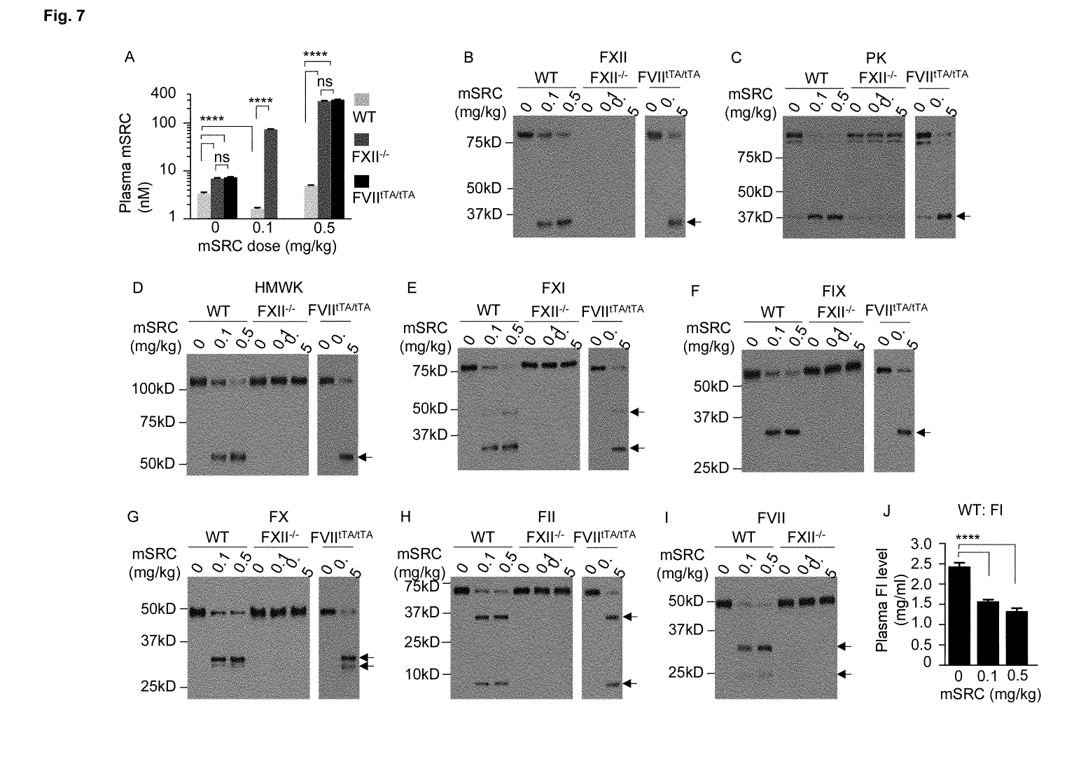

Incubation of mSRC with FVIIa (10 nM) and TF resulted in time-dependent and extensive mSRC fragmentation (FIG. 6C); thus, mSRC is also a substrate of FVIIa. The exact proteolytic sites in mSRC for FVIIa are not yet known. Average plasma level of endogenous mSRC was 3.4 nM in WT mice, but was 2.1- and 2.2-fold higher in FXII^{-/-} mice and FVII^{tTA/tTA} mice respectively (FIG. 7A). In WT mice, average plasma level of total mSRC was 49% and 147% of control at 6 h post i.p. injection of mSRC at 0.1 or 0.5 mg/kg, respectively (FIG. 7A). The effect of the low dose of mSRC on plasma level of total SRC is reminiscent of that seen with low dose of hPEPD as shown in FIG. 1A. At 6 h post mSRC injection described above, average plasma level of total mSRC was 45.2-58.2-fold higher in FXII^{-/-} mice and 62.2-fold higher in FVII^{tTA/tTA} mice (treated with mSRC only at 0.5 mg/kg) than in WT mice (FIG. 7A). Analysis of plasma samples showed that mSRC injection caused dose-related activation/cleavage of FXII, PK, HMWK, FXI, FIX, FX, FII, FI and FVII in WT mice, no change in any of the factors in FXII^{-/-} mice, and activation of all the factors, excluding FVII, in FVII^{tTA/tTa} mice (FIG. 7B-J). Unlike hPEPD and mPEPD, however, mSRC caused no FXII-independent activation of FX, FII and FVII. We did not measure FI cleavage in FXII^{-/-} mice and FVII^{tTA/tTA} mice. Collectively, our results show that the FXII-FVII pathway also detects and degrades mSRC.

### Example 2

This example demonstrates that the proteolytic pathway described in Example 1 is also responsible for degradation of amyloid beta proteins or peptides.

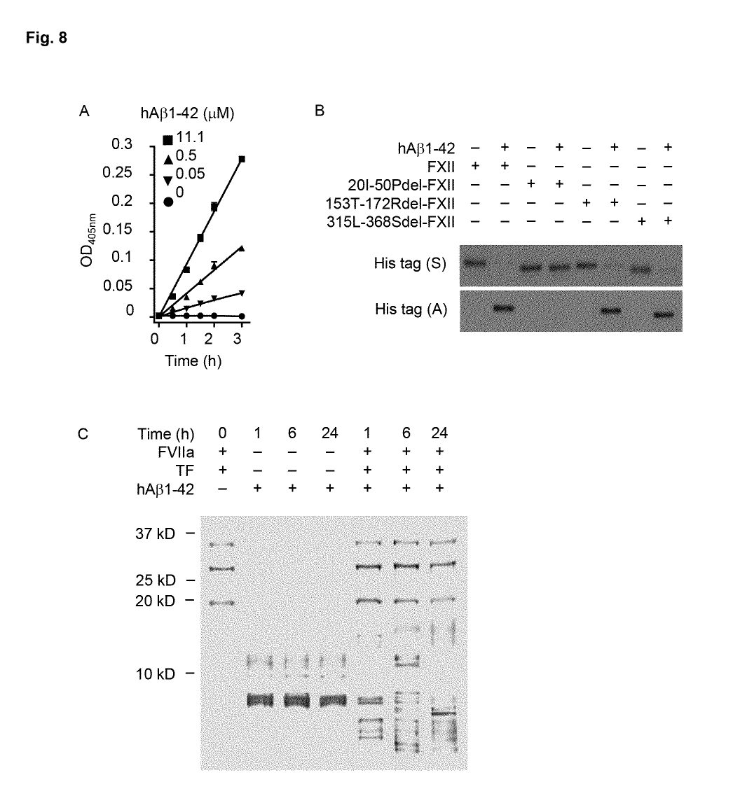

Degradation of plasma Aβ1-42 by the FXII-FVII proteolysis pathway. Human Aβ1-42 (hAβ1-42) also activates human FXII in a dose-dependent manner (FIG. 8A), but comparison of hAβ1-42 binding by FXII and its mutants showed that hAβ1-42 binds to FN2D (201-50P) in FXII (FIG. 8B). Incubation of hAβ1-42 with FVIIa (10 nM) and TF resulted in time-dependent and extensive hAβ1-42 fragmentation (FIG. 8C). The cleavage pattern indicates that hAβ1-42 is likely cleaved by FVIIa at all three sites where an arginine (residue #5) or a lysine (residues #16, 28) exists. The high molecular weight bands that formed after FVIIa treatment (FIG. 8C) may be aggregates of hAβ1-42 or its fragments, as Aβ1-42 is prone to aggregation.

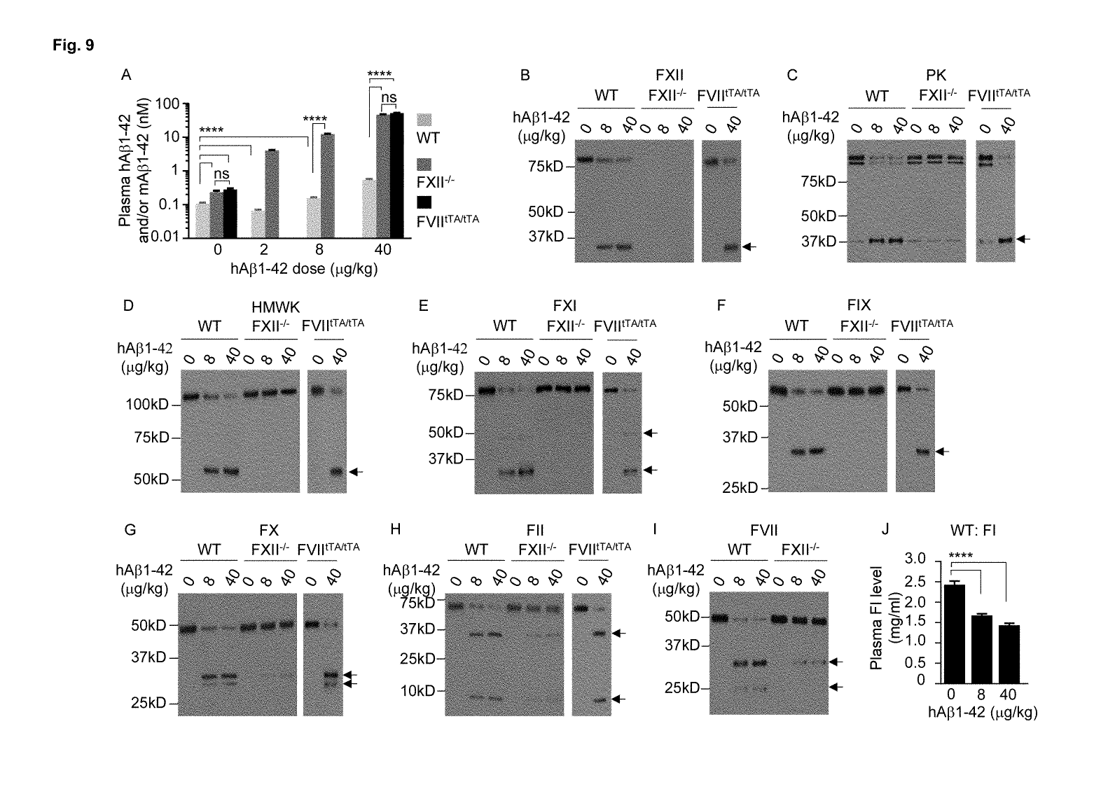

Average plasma level of endogenous mouse Aβ1-42 (mAβ1-42) was 0.1 nM in WT mice, but was 2.2-2.6-fold higher in FXII^{-/-} mice and FVII^{tTA/tTA} mice (FIG. 9A). In WT mice, at 6 h after i.p. injection of hAβ1-42 at 2 μg/kg, plasma level of total Aβ1-42 (mAβ1-42 plus hAβ1-42) decreased 39.3%, but it increased 1.5- and 5.1-fold after hAβ1-42 injection at 8 or 40 μg/kg (FIG. 9A). The drop in plasma level of total Aβ1-42 in response to the low dose of hAβ1-42 is reminiscent of that seen with low doses of hPEPD or mSRC as described before. Plasma levels of total Aβ1-42 were 62.7-94.1-fold higher in FXII^{-/-} mice and FVII^{tTA/tTA} mice than in WT mice under the same hAβ1-42 treatment, while the difference between FXII^{-/-} mice and FVII^{tTA/tTA} mice was not statistically significant (FIG. 9A).

hAβ1-42 caused the activation/cleavage of FXII, PK, HMWK, FXI, FIX, FX, FII, FI and FVII in WT mice, only slight activation of FX, FII and FVII in FXII^{-/-} mice, and activation of all the above factors, excluding FVII, in FVII^{tTA/tTA} mice (FIG. 9B-J). We did not measure FI cleavage in FXII^{-/-} mice and FVII^{tTA/tTA} mice. The slight FXII-independent activation of FX, FII and FVII by hAβ1-42 resembles that of hPEPD and mPEPD described before but did not contribute significantly to Aβ1-42 degradation. Our results show that Aβ1-42 is degraded exclusively by FVIIa in the plasma via the FXII-FVII pathway.

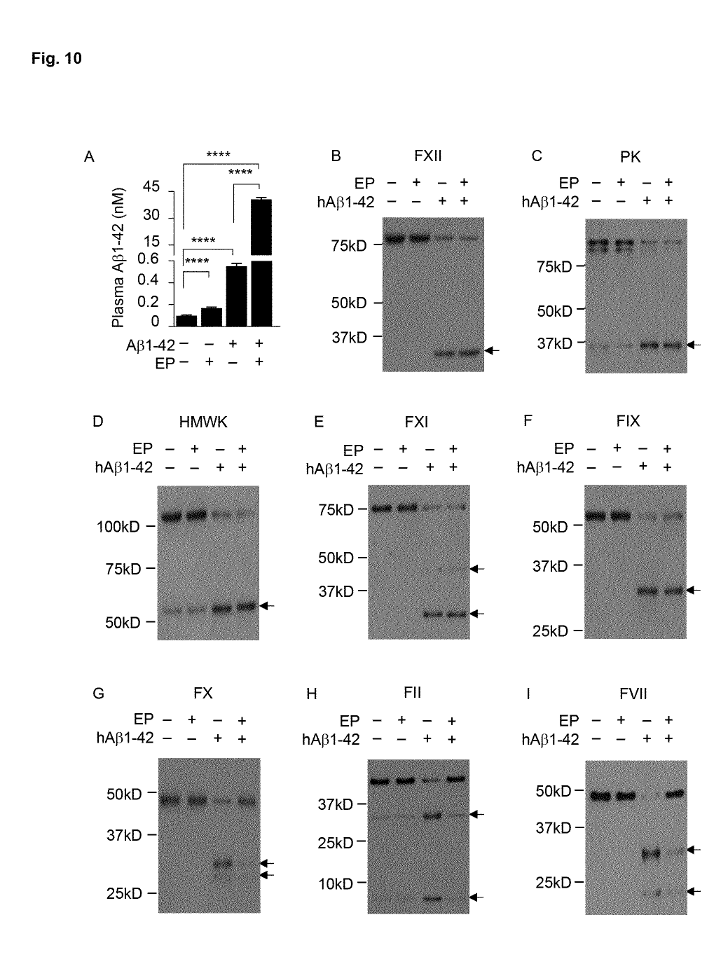

EP inhibits the degradation of plasma Aβ1-42. Given that Aβ1-42 is a key driver of AD, we sought to determine whether pharmacologically disrupting the FXII-FVII pathway blocks plasma Aβ1-42 degradation. We treated WT mice with vehicle or EP (2.5 mg/kg i.p.) once daily for 5 days, and 1 h after the last EP/vehicle dose, treated the mice i.p. with hAβ1-42 (40 μg/kg) or vehicle. Blood samples were collected from the mice at 6 h after hAβ1-42/vehicle treatment. Average plasma level of endogenous mAβ1-42 was 0.1 nM in control mice but was 1.7-fold higher in EP-treated mice (FIG. 10A). Plasma level of total Aβ1-42 increased 5.5-fold after treatment with hAβ1-42 alone but increased 406.0-fold after treatment with EP plus hAβ1-42 (FIG. 10A). Still, EP did not seem to completely block plasma Aβ1-42 degradation, based on the comparison of the above results with that in FXII^{-/-} mice and FVII^{tTA/tTA} mice that received the same hAβ1-42 treatment without EP as described before (FIG. 9A).

EP itself had no effect on any of the FXII-FVII pathway factors measured, but greatly inhibited the activation of FX, FII and FVII in hAβ1-42-treated mice (FIG. 10B-I). These results are similar to that shown in EP inhibition of plasma PEPD degradation as described before.

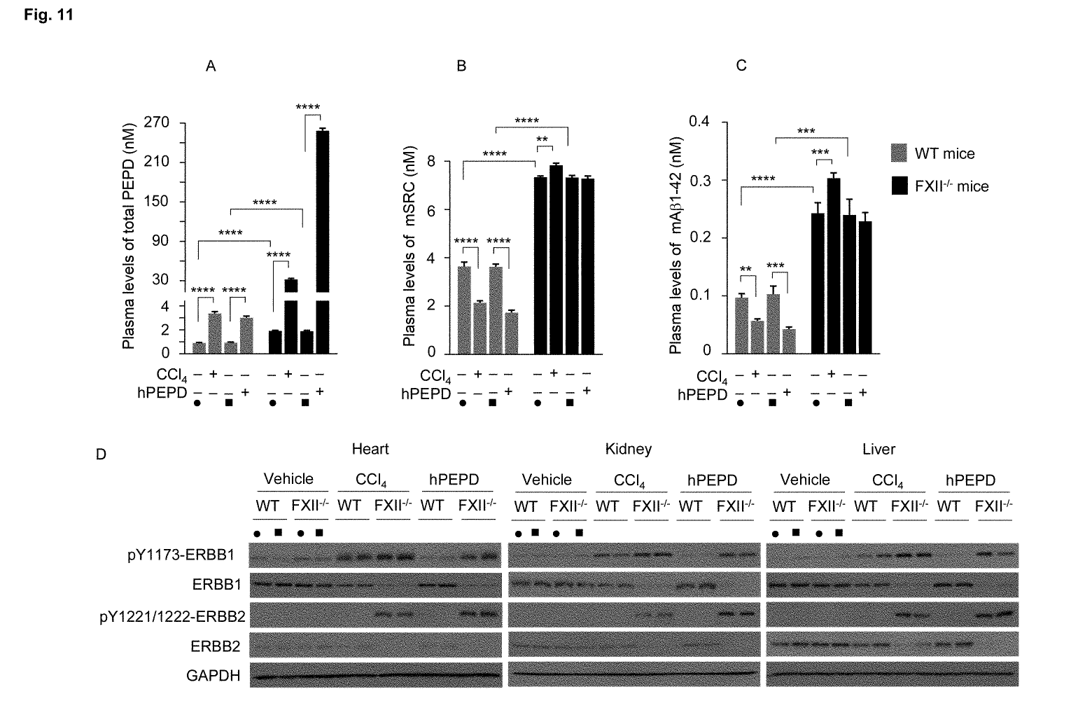

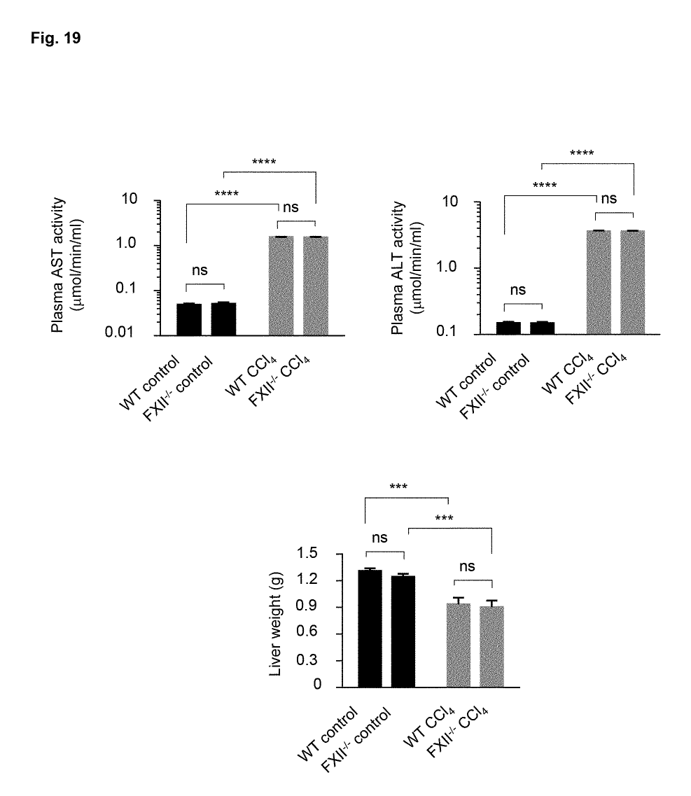

Activation of the FXII-FVII proteolysis pathway during tissue injury and its protective function. Carbon tetrachloride (CCl₄) causes liver damage and increases serum PEPD level, which presumably is due to PEPD release from the damaged tissues. CCl₄ treatment (0.5 g/kg) caused significant but similar liver damage between WT mice and FXII^{-/-} mice (FIG. 19). Plasma level of mPEPD was 2.1-fold higher in FXII^{-/-} mice than in WT mice before CCl₄ treatment, and at 24 h after CCl₄ treatment, plasma level of mPEPD increased 3.7-fold in WT mice but increased 16.7-fold in FXII^{-/-} mice (FIG. 11A). To corroborate the mPEPD results discussed above, we treated mice with hPEPD at 4 mg/kg. This dose of hPEPD was chosen so that plasma level of total PEPD in hPEPD-treated WT mice is similar to that in CCl₄-treated WT mice. At 24 h following hPEPD treatment, plasma level of total PEPD increased 3.3-fold in WT mice but increased 134.6-fold in FXII^{-/-} mice (FIG. 11A). Thus, whether PEPD is released from damaged tissues or introduced exogenously, it activates the FXII-FVII pathway, which in turn degrades the PEPD. In WT mice treated with CCl₄ or hPEPD, average plasma levels of mSRC and mAβ1-42 decreased 41.2-56.1% (FIGS. 11B and C). In contrast, in FXII^{-/-} mice, average plasma levels of mSRC and mAβ1-42 increased 6.7% and 24.9%, respectively, after CCl₄ treatment but remained unchanged after hPEPD treatment (FIGS. 11B and C). The small increase in plasma levels of mSRC and mAβ1-42 in CCl₄-treated FXII^{-/-} mice may be due to their release from the damaged tissues. Given that these substances are expressed in a variety of organs and tissues, PEPD in particular, the above results suggest that injury in various organs and tissues may activate the FXII-FVII pathway via release of these and other FXII activators to blood circulation. Furthermore, activation of this pathway by one protein may lead to degradation of multiple plasma proteins that are substrates of FVIIa.

PEPD is a ligand of ERBB1 and ERBB2 which are cell surface receptors. We sought to determine whether the FXII-FVII pathway may minimize the inhibitory effects of plasma PEPD (released from damaged tissues or entered exogenously) on the receptors. Compared to FXII^{-/-} mice, WT mice treated with CCl₄ or hPEPD showed not only lower plasma levels of PEPD (FIG. 11A) but also reduced changes in receptor tyrosine phosphorylation (measuring representative phosphorylation sites) and receptor depletion in various tissues (heart, kidney and liver) (FIG. 11D). Thus, the FXII-FVII pathway apparently attenuates the inhibitory effect of PEPD on ERBB1 and ERBB2 in normal tissues by degrading plasma PEPD.

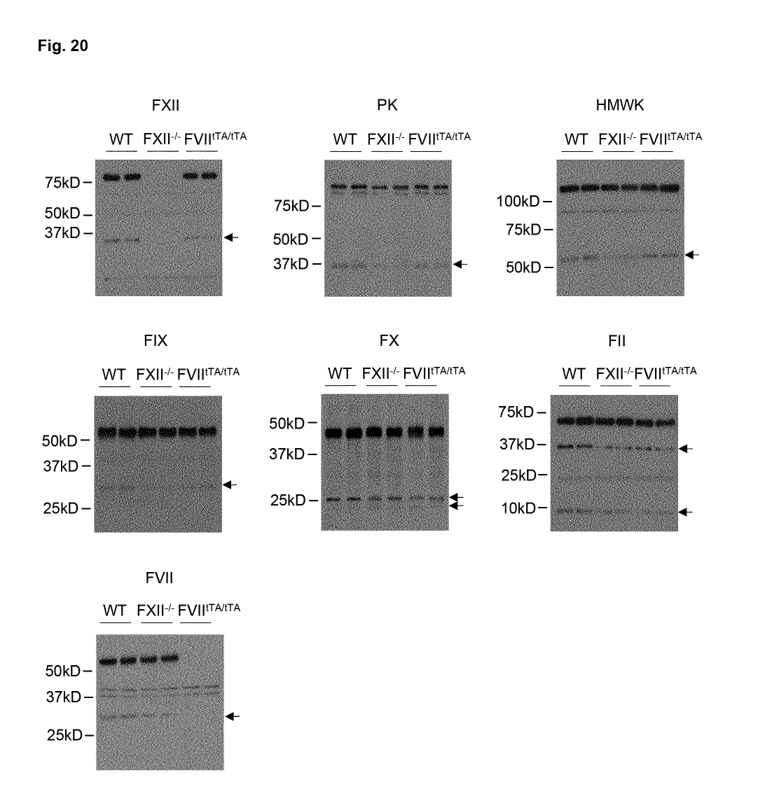

The FXII-FVII pathway for detecting and degrading PEPD, SRC and Aβ1-42 as well as its inhibition by EP are summarized in FIG. 12. FVIIa degrades PEPD, SRC and Aβ1-42 that are structurally diverse. PEPD and SRC activate FXII by binding to its PRD, whereas Aβ1-42 activates FXII by binding to its FN2D. Notably, the concentrations of these probes used for FXII activation in vitro are based on their concentrations detected in the plasma of FXII^{-/-} mice and FVII^{tTA/tTA} mice following treatment with them; the proteolysis pathway is disrupted in these mice, unlike WT mice in which this pathway was strongly activated under the same treatment conditions, leading to degradation of the probes. Other known activators of FXII interact with FN2D or FN1D in FXII. Thus, multiple domains in FXII mediate its activation. A large number of intracellular proteins bind to proline-rich motifs via their SH3, WW or EVH1 domains. Our SRC results suggest that other PRD-binding intracellular proteins may also engage the proteolysis pathway when released into blood circulation. On the other hand, it is possible that other mechanisms may also play a role in clearing these and other proteins and peptides from the circulation, such as renal elimination, hepatic elimination, binding to matrix in various tissues as well as cellular uptake and degradation. Notably, there is only slight activation of various factors of the FXII-FVII pathway in un-treated mice (FIG. 20), suggesting that this pathway is not significantly activated under normal conditions; detection of such activation was somewhat difficult, as there appeared to be a slight activation of the factors following blood draw before the anticoagulant effect of EDTA kicked in.

Our results indicate that the FXII-FVII pathway may contribute to tissue homeostasis by eliminating unwanted or harmful proteins and peptides from plasma, whereas anticoagulants like EP may elevate plasma level of these substances by disrupting the proteolysis pathway. Notably, no physiological function in the blood is known for PEPD, SRC and Aβ1-42, and Aβ1-42 is even harmful, being a key driver of AD. However, our results also show that activation of this pathway leads to significant cleavage of FI, which may increase blood clot risk, although there was no sign of blood clotting in the mice in the present study. Moreover, activation of this pathway may also lead to bradykinin liberation from HMWK and complement activation via β-FXIIa and kallikrein, potentially impacting vascular physiology, immune response and inflammation.

Our finding that plasma Aβ1-42 is detected and degraded by the FXII-FVII pathway provides insight into AD. Our results show that activation of FXII leads to FVII activation and degradation of Aβ1-42 by FVIIa. Aβ1-42 and Aβ1-40 are the main components of senile plaque, one of the hallmarks of AD pathology, and are considered key drivers of AD. Like Aβ1-42, Aβ1-40 is generated from proteolytic cleavage of cell-membrane-bound Aβ precursor protein by secretases and is also present in the plasma at low levels. We show that Aβ1-40 is also degraded by the FXII-FVII pathway. The fact that total plasma levels of Aβ1-42 are 63-95-fold higher in mice deficient in either FXII or FVII or in EP-pretreated WT mice than in EP-untreated WT mice following hAβ1-42 injection (FIGS. 9A and 10A) is a clear evidence that the newly discovered plasma proteolysis pathway plays a pivotal role in plasma Aβ1-42 clearance.

Moreover, our finding that EP inhibits plasma Aβ1-42 and Aβ1-40 degradation may provide a novel approach for development of AD biomarkers. Blood-based biomarkers of AD are an unmet medical need. Plasma Aβ level correlates poorly with brain plaque burden and is not currently considered an AD biomarker (Blennow et al., Nat Rev Neurol 2010; 6(3): 131-144; Hansson et al., J Alzheimers Dis 2012; 28(1): 231-238; Rissman et al., J Neural Transm 2012; 119(7): 843-850; Toledo et al., Alzheimers Res Ther 2013; 5(2):8). Our results indicate that temporary inhibition of the FXII-FVII proteolysis pathway, e.g., using EP, may enable detection of high plasma levels of Aβ1-42, Aβ1-40 and other Aβ peptides in AD, and that such an approach can enable development of plasma Aβ as an AD biomarker for disease detection as well as for assessment of disease progression and response to treatment.

The discovery of the FXII-FVII proteolysis pathway may also have important implications for developing certain protein therapeutics. Many therapeutic proteins have short plasma half-life, and current approaches are aimed at slowing their removal via biliary, hepatic or renal elimination. Our findings indicate that some of these proteins may engage the FXII-FVII pathway and that inhibiting this pathway may increase their retention in plasma. As a case in point, EP inhibits PEPD degradation in vivo and allowed hPEPD dose to be reduced by at least 50 fold without decreasing its plasma concentration required for inhibition of ERBB2-driven tumors in mice. Moreover, combination of EP with hPEPD may also make hPEPD a safer antitumor agent, as EP may minimize the stimulating effect of hPEPD on blood coagulation.

TABLE 1
The sequences of primers used for cloning, site-directed mutations and partial
deletions of His-tagged human FXII

| Target vector | Primer | Sequence |
|---|---|---|
| pCMV6-XL5-FXII | For | 5′-GAATTCgaccaacggacggacgc-3′ |
| | Rev | 5′-GTCGAC ggaaacggtgtgctccc-3′ |
| pCMV6-XL5-FXII-6XHis (C-terminal | For | 5′-tagattgcggccgcggcatcatcaccatcaccattaatcatagctgtacctg-3′ |
| 6XHis insertion) | Rev | 5′-caggaaacagctatgattaatggtgatggtgatgatgccgcggccgcaatcta-3′ |
| pCMV6-XL5-20I-50Pdel-FXII-6XHis | For | 5′-ggagtcaacactttcgttccagtaccaccggc-3′ |
| | Rev | 5′-gccggtggtactggaacgaaagtgttgactcc-3′ |
| pCMV6-XL5-153T-172Rde1-FXII- | For | 5′-aagaatgagatatggtatagactggccagccaggc-3′ |
| 6XHis | Rev | 5′-gcctggctggccagtctataccatatctcattctt-3′ |
| pCMV6-XL5-315L-368Sde1-FXII- | For | 5′-tcccctaggcttcatgtcccatcgatgacccg-3′ |
| 6XHis | Rev | 5′-cgggtcatcgatgggacatgaagcctagggga-3′ |
| pCMV6-XL5-6Psdel-FXII-6XHis | For | 5′-atgtcccactcatgacgacccggacccc-3′ |
| | Rev | 5′-ggggtccgggtcgtcatgagtgggacat-3′ |
| pCMV6-XL5-13Ps > 13As-FXII- | For 1 | 5′-catggccgcgcaggcggcagcggcgaaggctcaggccac-3′ |
| 6XHis^{a} | For 2 | 5′-cgacccggaccgcggctcagtcccag-3′ |
| | For 3 | 5′-cagtcccagaccgcgggagccttgc-3′ |
| | For 4 | 5′-cgggagccttggcggcgaagcgg-3′ |
| | For 5 | 5′-aagcgggagcaggcggcncectgacca-3′ |
| | For 6 | 5′-accaggaacggcgcactgagctgcg-3′ |

^{a}Multiple sets of primers were used to generate this FXII mutant.

### Example 3

This example demonstrates that hAβ1-40 activates FXII and is degraded by FVIIa. Human amyloid β1-40 (hAβ1-40) was purchased from Sigma (A1075). Assay protocols and reagents are as described in Examples 1 and 2. hAβ1-40 (0, 0.05, 0.5 μM) was incubated with human FXII (0.97 nM) in ZnCl₂-containing PBS at room temperature; FXII activation was measured by a chromogenic assay. Each value is mean±SD (n=3). (B) hAβ1-40 (2.2 μM) was incubated alone or with FVIIa (10 nM) plus human TF (10 nM) in CaCl₂-containing PBS at room temperature for indicated times; after removing the supernatant fraction, each incubation tube was rinsed with a small volume of 2% SDS, to dissolve potential aggregates, and the rinse was mixed with the supernatant fraction, separated by SDS-PAGE, and stained by silver. FVIIa and TF were incubated without hAβ1-40, as a control.

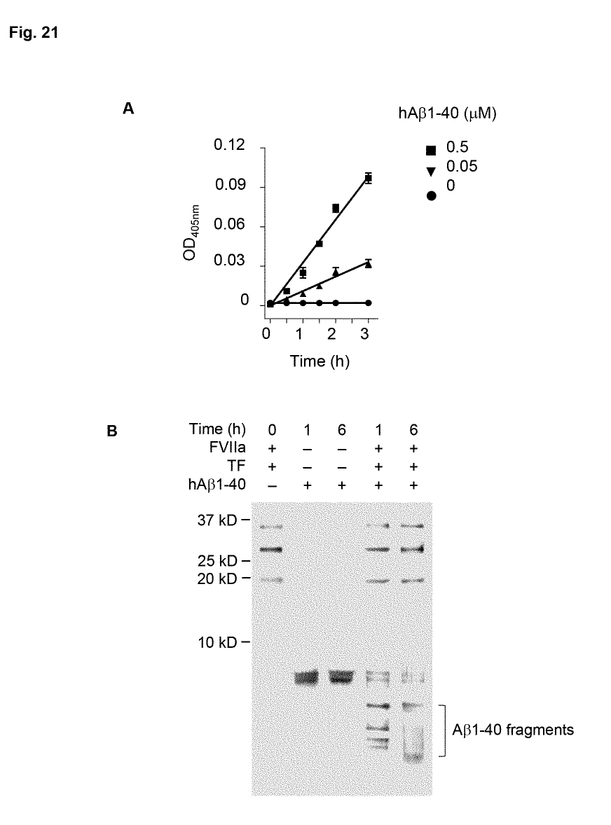

FIG. 21A shows that hAβ1-40 activates FXII in a dose- and time-dependent manner. FIG. 21B shows that activated factor VII (FVIIa) together with tissue factor (TF) degrades hAβ1-40. Thus, hAβ1-40 resembles hAβ1-42 in activation of FXII and its degradation by FVIIa.

### Example 4

Male C57BL/6 mice (7-8 weeks of age) were purchased from Taconic and were treated with vehicle (PBS) and EP (0.5 mg/kg) i.p. once daily for 5 days. One hour after the fifth dose, hAβ1-40 (40 μg/kg) was administered i.p. to the mice, and 6 h later, blood was collected from the mice by cardiac puncture at the time of sacrifice by carbon dioxide. Blood was collected into K3 EDTA-containing tubes (Multivette 600 from Sarstedt). EP and hAβ1-40 were each given in PBS (0.1 ml per 20 g body weight). Plasma levels of hAβ1-40 were measured by ELISA. Briefly, 96-well ELISA plates were coated with an hAβ1-40-detecting mouse monoclonal antibody at 4° C. overnight. The plates were washed three times with phosphate buffered saline with tween-20 (PBST) and the coated wells were blocked by incubation with 200 μl/well of 1% BSA in PBS for at least 2 h at room temperature (RT). After another round of wash with PBST, the plates were incubated with appropriately diluted hAβ1-40 standard or plasma samples (100 μl/well) for 2 h at RT. The plates were then washed with PBST and incubated with an anti-hAβ1-40 rabbit polyclonal antibody for 2 h at RT. The plates were washed again with PBST, and each well was incubated with a goat anti-rabbit IgG-HRP conjugate (100 μl) for 1 h at RT. After yet another round of wash with PBST, each well was incubated with 100 μl of HRP substrate 3,3′,5,5′-tetramethylbenzedine (Cell Signaling, 7004). Upon adequate color development, 100 μl of stop solution (Cell Signaling, 7002) was added to each well, and absorbance at 450 nm was promptly recorded by a microtiter plate reader.

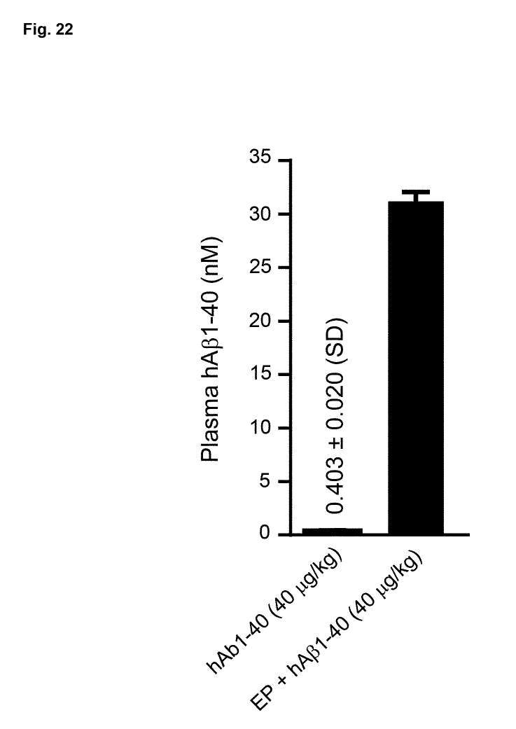

As shown in FIG. 22, without EP pretreatment, average plasma hAβ1-40 concentration is 0.403 nM, but EP pretreatment elevates average plasma hAβ1-40 concentration by 77.4 fold. This shows that EP blocks hAβ1-40 degradation in vivo, besides blocking hAβ1-42 degradation in vivo as shown before.

### Example 5

B6.Cg-Tg(PDGFB-APPSwlnd) 20 Lms/2 JMjax mice, also known as J20 mice, were purchased from Jackson Laboratory. J20 mice express a mutant form of the human Aβ precursor protein (APP) bearing both the Swedish (K670N/M671L) and the Indiana (V717F) mutations (APPSwlnd) (Mucke et al., J Neurosci, 2000, 20, 4050-4058). Neural expression of the transgenic insert is directed by the human platelet-derived growth factor beta polypeptide (PDGFB) promoter. J20 mice in C57BL/6 background (male hemizygotes) were bred to wild type (WT) female C57BL/6J mice (also purchased from Jackson Laboratory). F1 mice were genotyped by PCR at weaning to identify the hemizygotes and the non-transgenic littermates. Mice at age of 1 month and 3 months were treated once daily with vehicle or EP (0.5 mg/kg) i.p for 5 days. EP was given in PBS (0.1 ml per 20 g body weight). Blood was collected from the mice at 6 h after the last PBS/EP dose by cardiac puncture at the time of sacrifice by carbon dioxide. Blood was collected into K3 EDTA-containing tubes (Multivette 600 from Sarstedt).

Total plasma levels of Aβ1-40 and Aβ1-42 were measured by ELISA. Briefly, 96-well ELISA plates were coated with an anti-Aβ1-42 monoclonal antibody (sc-374527) at 0.25 μg/100 μl/well at 4° C. overnight. The plates were washed three times with phosphate buffered saline tween-20 (PBST) and the coated wells were blocked by incubation with 200 μl/well of 1% BSA in PBS for at least 2 h at RT. After another round of wash with PBST, the plates were incubated with appropriately diluted hAβ1-42 standard or plasma samples (100 μl/well) for 2 h at RT. The plates were then washed with PBST and incubated with an anti-Aβ1-42 rabbit polyclonal antibody (sc-9129) at 100 μl/well for 2 h at RT. The plates were washed again with PBST, and each well was incubated with a goat anti-rabbit IgG-HRP conjugate (100 μl) for 1 h at RT. After yet another round of wash with PBST, each well was incubated with 100 μl of HRP substrate 3,3′,5,5′-tetramethylbenzedine (Cell Signaling, 7004). Upon adequate color development, 100 μl of stop solution (Cell Signaling, 7002) was added to each well, and absorbance at 450 nm was promptly recorded by a microtiter plate reader. Notably, the above ELISA assay detects both Aβ1-40 and Aβ1-42, both the human forms and mouse forms.

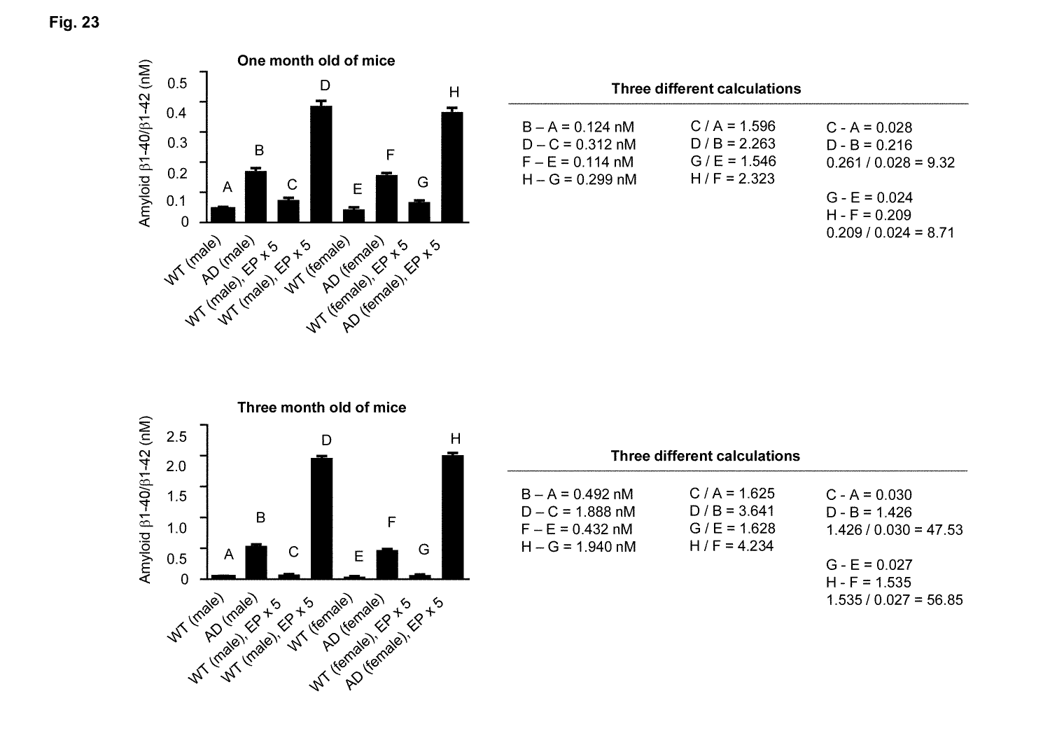

Total plasma levels of Aβ1-40 and Aβ1-42 in WT mice are low (approximately 0.45 nM), are similar between male mice and female mice, and are also similar at 1 month and 3 months of age (FIG. 23). Total plasma levels of Aβ1-40 and Aβ1-42 in AD mice, while not different between male mice and female, are significantly higher than in WT mice, and increase in a time-dependent manner (FIG. 23). EP has limited effect on total plasma levels of Aβ1-40 and Aβ1-42 in WT mice, causing about 1.6 fold increase at both 1 month and 3 months of age. In contrast, EP markedly increases total plasma levels of Aβ1-40 and Aβ1-42 in AD mice, causing about 2.3 fold increase at 1 month of age and 3.6-4.2 fold increase at 3 month of age (FIG. 23). Most notably, the ratio of EP-induced increase in total plasma levels of Aβ1-40 and Aβ1-42 in AD mice to WT mice is 8.71-9.32 at 1 month of age and 47.53-56.85 at 3 month of age (FIG. 23). These results show that EP blocks the degradation of Aβ1-40 and Aβ1-42 that are released from brain to peripheral circulation in AD mice.

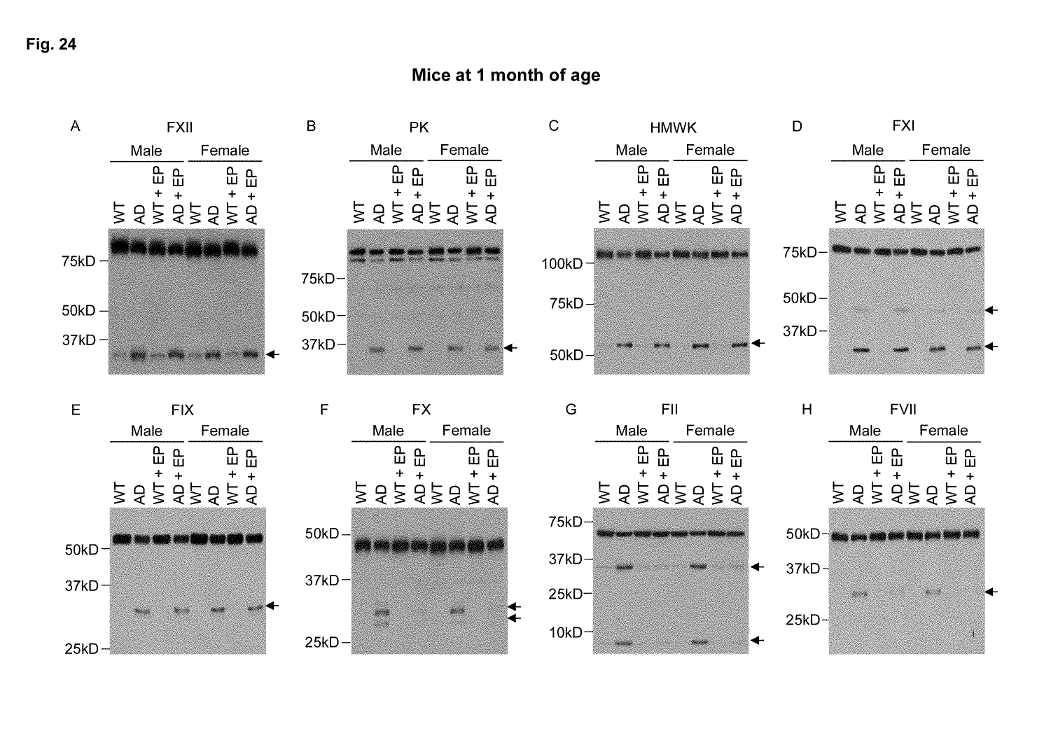

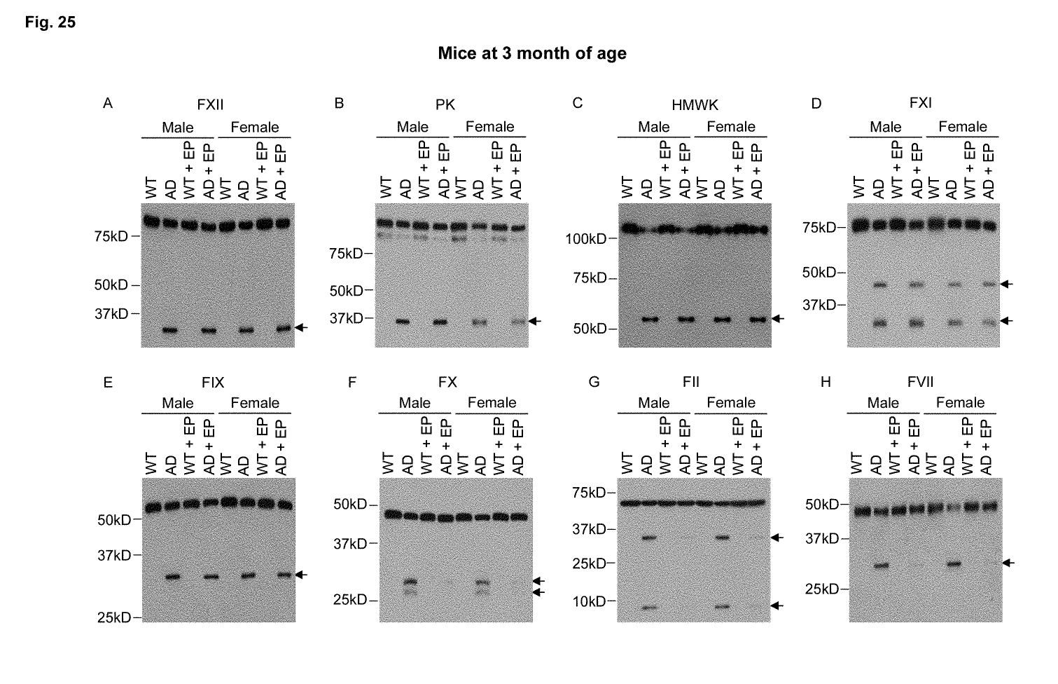

Coagulation factors are cleaved or activated in AD mice, including FXII, PK, HMWK, FXI, FIX, FX, FII and FVII, and the cleavage/activation is more significant at 3 months of age than at 1 month of age (FIG. 24 and FIG. 25), reflecting increased plasma concentration of Aβ1-40 and Aβ1-42. EP inhibits the activation of FX, FII and FVII (FIG. 24 and FIG. 25).

### Example 6

In addition to EP, we measured the effects of 3 clinically used oral anticoagulants on plasma Aβ degradation (using hAβ1-42 as an example), including rivaroxaban (a direct FXa inhibitor; Roehrig et al., J Med Chem, 2005, 48, 5900-5908), dabigatran (a direct FIIa inhibitor; van Ryn et al., Thromb Haemost, 2010, 103, 1116-1127), and warfarin (inhibiting the synthesis of vitamin K-dependent factors, including FII, FVII, FIX and FX; Whitlon et al., Biochemistry, 1978, 17, 1371-1377). Based on the literature data of their effects on coagulation in mice (Zhou et al., Stroke, 2013, 44, 771-778; Pfeilschifter et al., Stroke, 2011, 42, 1116-1121; Sato et al., Jpn Pharmacol, 1998, 78, 191-197; Sparkenbaugh et al., Blood, 2014, 123, 1747-1756; DeFeo et al., Cancer Biol Ther, 2010, 10, 1001-1008), male C57BL/6 mice (7-8 weeks of age), purchased from Taconic, were treated with vehicle, rivaroxaban (10 or 20 mg/kg), dabigatran (22.5 or 45 mg/kg) or warfarin (1 or 3 mg/kg) by oral intubation once daily for 5 days. One hour after the fifth dose, hAβ1-42 (40 μg/kg) was administered i.p. to the mice, and 6 h later, blood was collected from the mice by cardiac puncture at the time of sacrifice by carbon dioxide. Blood was collected into K3 EDTA-containing tubes (Multivette 600 from Sarstedt). Plasma levels of hAβ1-42 were measured by ELISA. Rivaroxaban, dabigatran (dabigatran elexilate) and warfarin (warfarin sodium tablets) were from Bristol-Myers-Squibb, Combi-Blocks, and Advanced Chemblocks, respectively. Rivaroxaban was dissolved in 10% ethanol, 40% Solutol HS15 (Sigma) and 50% water and was administered to mice in 0.2 ml volume per 20 g body weight. Dabigatran was dissolved in 2% dimethyl sulfoxide in soy oil (Sigma) and was also administered to mice in 0.2 ml volume per 20 g body weight. Warfarin was dissolved in water and was administered to mice in 0.1 ml volume per 20 g body weight. Notably, the top doses of rivaroxaban and dabigatran are the maximal soluble doses, whereas the top dose of warfarin is the maximal tolerated dose.

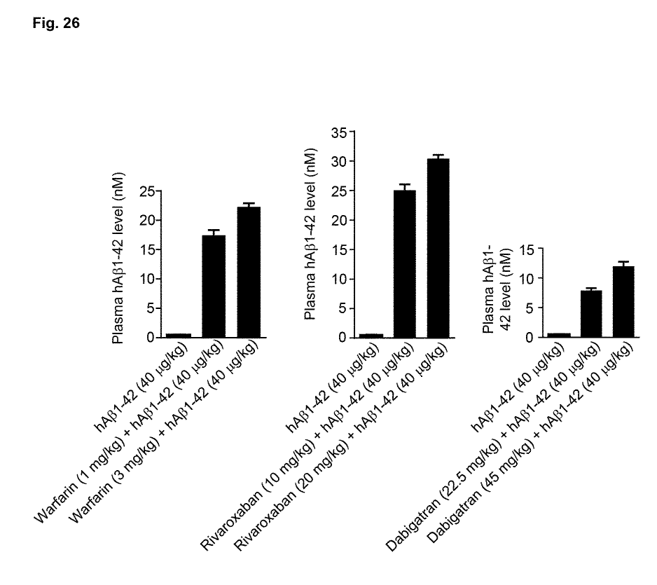

As shown in FIG. 26, average plasma level of hAβ1-42 is 0.53-0.56 nM in mice 6 h after injection of hAβ1-42 at 40 μg/kg. Pretreatment with warfarin at 1 and 3 mg/kg elevated plasma levels of hAβ1-42 by 33.2 fold and 42.4 fold, respectively. Pretreatment with rivaroxaban at 10 and 20 mg/kg elevated plasma levels of hAβ1-42 by 47.2 fold and 55.5 fold, respectively. Pretreatment with dabigatran at 22.5 and 45 mg/kg elevated plasma levels of hAβ1-42 by 14.1 fold and 21.3 fold, respectively. Thus, among the three agents, rivaroxaban is the most effective and dabigatran is the least effective in elevating plasma hAβ1-42 level. This is consistent with the fact that rivaroxaban is a direct FXa inhibitor which is believed to more completely block activation of FVII via FXII by Aβ peptides, dabigatran is a direct FIIa inhibitor which is believed to only partially block FVII activation via FXII by Aβ peptides, and warfarin indirectly inhibits multiple coagulation proteases (FII, FVII, FIX and FX). However, rivaroxaban is not as effective as EP in elevating plasma level of hAβ1-42. EP is known to inhibit FIXa, FXa and FIIa by activating antithrombin III.

1. A method for detecting Aβ peptides in blood in an individual comprising:
a) administering to the individual a composition comprising an inhibitor of a blood proteolytic pathway in an amount sufficient to inhibit one or more steps of the pathway;
b) after a suitable period of time, collecting a blood sample from the individual; and
c) determining the amount of one or more Aβ peptides in the sample or a fraction thereof.

2. The method of claim 1, wherein the inhibitor of the blood proteolytic pathway is an anticoagulant.

3. The method of claim 2, wherein the anti-coagulant is a low molecular weight heparin.

4. The method of claim 3, wherein the low molecular weight heparin is selected from the group consisting of ardeparin, bemiparin, enoxaparin, certoparin, dalteparin, nadroparin, reviparin, parnaparin and tinzaparin.

5. The method of claim 2, wherein the anticoagulant is apizaban, rivaroxaban, dadigatran, warfarin and heparin.

6. The method of claim 1, wherein the Aβ peptide is Aβ1-40 and/or Aβ1-42.

7. The method of claim 1, further comprising the step of comparing the levels of Aβ peptide in the blood sample to a reference.

8. The method of claim 1, further comprising the step of preparing plasma from blood prior to determining the amount of Aβ peptide.

9. The method of claim 1, wherein blood is collected from the individual after at least 5 minutes after administration of the inhibitor.

10. A method for identifying increased amyloid beta plaques in an individual comprising the steps of:
a) administering to the individual an inhibitor of a blood proteolytic pathway in an amount sufficient to inhibit one or more steps of the pathway;
b) after a suitable period of time during the course of administration or after termination of the administration, collecting a blood sample from the individual; and
c) determining the amount of a Aβ peptide in the sample;
wherein an increase in the amount of the Aβ peptide in the sample from the individual compared to a reference sample is indicative of an increase in amyloid beta plaques in an individual.

11. The method of claim 10, wherein the inhibitor of the blood proteolytic pathway is an anticoagulant.

12. The method of claim 11, wherein the anticoagulant is a low molecular weight heparin.

13. The method of claim 12, wherein the low molecular weight heparin is selected from the group consisting of ardeparin, bemiparin, enoxaparin, certoparin, dalteparin, nadroparin, reviparin, parnaparin and tinzaparin.

14. The method of claim 11, wherein the anticoagulant is selected from the group consisting of apizaban, rivaroxaban, dadigatran, warfarin and heparin.

15. The method of claim 10, wherein the Aβ peptide is Aβ1-40 and/or Aβ1-42.

16. The method of claim 1, wherein the individual is diagnosed with, or has, or is at risk of developing Alzheimer's disease.

17. The method of claim 10, wherein the increased amyloid beta plaques are in the brain.

18. A method for monitoring the effect of an Alzheimer's Disease (AD) treatment in an individual comprising the steps of:
a) administering to an individual, who is being treated for AD, an inhibitor of a blood proteolytic pathway in an amount sufficient to inhibit one or more steps of the pathway;
b) after a suitable period of time during the course of administration of the inhibitor or after termination of the administration of the inhibitor, collecting a blood sample from the individual;
c) determining the level of one or more Aβ peptides in the sample, plasma or serum prepared from the sample; and
d) comparing the level of the one or more Aβ peptides in the sample with a reference level to identify of the effectiveness of the AD treatment.

19. The method of claim 18, wherein the inhibitor of the blood proteolytic pathway is enoxaparin and the Aβ peptide is Aβ1-40 and/or Aβ1-42.

20. A kit for detecting the level of an Aβ peptide in the blood of an individual comprising:
a) one or more doses of a composition comprising an inhibitor of a blood proteolytic pathway; and
b) reagents for detecting one or more Aβ peptides in a blood sample or a fraction thereof; and
c) optionally, instructions for one or more of the following: administration and storage of the inhibitor, and use of regents for detecting Aβ peptide.

21. The method of claim 10, wherein the individual is diagnosed with, or has, or is at risk of developing Alzheimer's disease.


SEQUENCE LISTING (18 sequences)

SEQ ID NO: 1 - DNA, 23 bp, artificial sequence
  Primer
gaattcgaccaacggacggacgc

SEQ ID NO: 2 - DNA, 23 bp, artificial sequence
  Primer
gtcgacggaaacggtgtgctccc

SEQ ID NO: 3 - DNA, 53 bp, artificial sequence
  Primer
tagattgcggccgcggcatcatcaccatcaccattaatcatagctgtttcctg

SEQ ID NO: 4 - DNA, 53 bp, artificial sequence
  Primer
caggaaacagctatgattaatggtgatggtgatgatgccgcggccgcaatcta

SEQ ID NO: 5 - DNA, 32 bp, artificial sequence
  Primer
ggagtcaacactttcgttccagtaccaccggc

SEQ ID NO: 6 - DNA, 32 bp, artificial sequence
  Primer
gccggtggtactggaacgaaagtgttgactcc

SEQ ID NO: 7 - DNA, 35 bp, artificial sequence
  Primer
aagaatgagatatggtatagactggccagccaggc

SEQ ID NO: 8 - DNA, 35 bp, artificial sequence
  Primer
gcctggctggccagtctataccatatctcattctt

SEQ ID NO: 9 - DNA, 32 bp, artificial sequence
  Primer
tcccctaggcttcatgtcccatcgatgacccg

SEQ ID NO: 10 - DNA, 32 bp, artificial sequence
  Primer
cgggtcatcgatgggacatgaagcctagggga

SEQ ID NO: 11 - DNA, 28 bp, artificial sequence
  Primer
atgtcccactcatgacgacccggacccc

SEQ ID NO: 12 - DNA, 28 bp, artificial sequence
  Primer
ggggtccgggtcgtcatgagtgggacat

SEQ ID NO: 13 - DNA, 39 bp, artificial sequence
  Primer
catggccgcgcaggcggcagcggcgaaggctcaggccac

SEQ ID NO: 14 - DNA, 26 bp, artificial sequence
  Primer
cgacccggaccgcggctcagtcccag

SEQ ID NO: 15 - DNA, 25 bp, artificial sequence
  Primer
cagtcccagaccgcgggagccttgc

SEQ ID NO: 16 - DNA, 23 bp, artificial sequence
  Primer
cgggagccttggcggcgaagcgg

SEQ ID NO: 17 - DNA, 28 bp, artificial sequence
  Primer
aagcgggagcaggcggcttccctgacca

SEQ ID NO: 18 - DNA, 24 bp, artificial sequence
  Primer
accaggaacggcgcactgagctgc